SEQUENCE LISTING (17 sequences)

SEQ ID NO: 1 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 390 aa, Sus scrofa scrofa
MPPSGLRLLPLLLPLLWLLVLTPGRPAAGLSTCKTIDMELVKRKRIEAIRGQILSKLRLASPPSQGDVPPGPLPEAVLALYNSTRDRVAGESVEPEPEPEADYYAKEVTRVLMLESGNQIYDKFKGTPHSLYMLFNTSELREAVPEPVLLSRAELRLLRLKLKVEQHVELYQKYSNDSWRYLSNRLLAPSDSPEWLSFDVTGVVRQWLTRREAIEGFRLSAHCSCDSKDNTLHVEINGFNSGRRGDLATIHGMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 3 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 4 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 5 - protein, 18 aa, Rattus rattus
MKWVTFLLLLFISGSAFS

SEQ ID NO: 6 - DNA, 36 bp, Cricetulus griseus
gcgatacccgggtataccatggccacctcagcaagt

SEQ ID NO: 7 - DNA, 33 bp, Cricetulus griseus
cgggtgttcgaattagtttttgtattggaaggg

SEQ ID NO: 8 - DNA, 56 bp, Homo sapiens
ggttctgccttttctcaccaccatcaccaccaccatcatctgtccacctgcaagac

SEQ ID NO: 9 - DNA, 30 bp, Homo sapiens
tagtctcgagttatcagctgcacttgcagg

SEQ ID NO: 10 - DNA, 45 bp, Rattus rattus
aaagggggatccgccaccatgaagtgggtaacctttctcctcctc

SEQ ID NO: 11 - DNA, 45 bp, Rattus rattus
agaaaaggcagaaccggagatgaagaggaggaggagaaaggttac

SEQ ID NO: 12 - DNA, 39 bp, Sus scrofa scrofa
cgcctcagtgcccactgttcctgtgacagcaaagataac

SEQ ID NO: 13 - DNA, 39 bp, Sus scrofa scrofa
gttatctttgctgtcacaggaacagtgggcactgaggcg

SEQ ID NO: 14 - DNA, 33 bp, Sus scrofa scrofa
ggatccctgtccacctccaagaccatcgacatg

SEQ ID NO: 15 - DNA, 33 bp, Sus scrofa scrofa
catgtcgatggtcttggaggtggacagggatcc

SEQ ID NO: 16 - protein, 20 aa, Artificial Sequence
  Synthetic construct
HHHHHHHHLSTSKTIDMELV

SEQ ID NO: 17 - protein, 20 aa, Sus scrofa scrofa
ALDTNYCFSSTEKNCCVRQL

(19) United States
(12) Patent Application Publication
(10) Pub. No.: US 2011/0165623 A1
(43) Pub. Date: Jul. 7, 2011

(54) Compositions and Methods for the High Efficiency Expression of the Transforming Growth Factor-Beta Supergene Family

(75) Inventors: Peter Daopin Sun, Derwood, MD (US); Zongcheng Zou, Rockville, MD (US)

(73) Assignee: THE GOVERNMENT OF THE U.S. AS REPRESENTED BY THE SECRETARY, DEPT. OF HEALTH & HUMAN SERVICES, Rockville, MD (US)

(21) Appl. No.: 10/585,499

(22) PCT Filed: Jan. 6, 2005
(86) PCT No.: PCT/US05/00378

§ 371 Date: Jul. 6, 2006

Related U.S. Application Data
(60) Provisional application No. 60/534,379, filed on Jan. 6, 2004.
(60) Provisional application No. 60/575,839, filed on Jun. 2, 2004.

(51) Int. Cl. C12P 21/02; C07H 21/00; C12N 5/10; C12N 15/63; C07H 21/02; C07K 19/00; C12N 5/071

(52) U.S. Cl. 435/69.7; 536/23.4; 435/325; 435/320.1; 530/399; 435/69.1; 435/360

(57) ABSTRACT

Novel compositions and methods for the high efficiency production of the transforming growth factor-beta (TGFβ) supergene family of peptide growth factors are provided.

### CROSS-REFERENCE TO RELATED APPLICATIONS

This application claims priority to U.S. Provisional Patent Application Ser. No. 60/534,379, filed Jan. 6, 2004 and 60/575,839, filed Jun. 2, 2004.

### GOVERNMENTAL RIGHTS

This invention was funded by the National Institute of Allergy and Infectious Diseases at the National Institutes of Health. The United States Government has certain rights to this invention.

### FIELD

The present invention relates to compositions and methods for the high efficiency production of the transforming growth factor-β (TGFβ) supergene family members.

### BACKGROUND

Transforming growth factor-β1 (TGFβ1) was first isolated from human placenta in 1983 (Frolik et al., 1983, Proc. Natl. Acad. Sci. U.S. A 80:3676-3680). There are currently five distinct isoforms of TGFβ sharing 64-82% identity; three of the five, TGFβ1, -β2 and -β3, are expressed in mammalian tissues (Govinden et al., 2003, Pharmacol. Ther. 98:257-265). TGFβ1 belongs to a supergene family of over 40 different related proteins, presumed to be derived from a common ancestor gene (Kingsley et al., 1994, Genes Dev. 8:133-146). TGFβ1 is the prototype of the supergene family.

Although TGFβ1 was initially described as a factor that caused rat kidney fibroblasts to proliferate (Roberts et al., 1981, Proc. Natl. Acad. Sci. U.S.A. 78:5339-5343), it is now known that TGFβ1 is a multifunctional cytokine with both stimulatory and inhibitory effects on a wide range of cells (Lawrence, 2001, Mol. Cell. Biochem. 219:163-170). The TGFβs 1, 2, and 3 are involved in normal tissue development such as wound healing, angiogenesis, hematopoiesis, mammary gland development, bone metabolism, and skin formation. The TGFβs 1, 2, and 3 are also associated with multiple pathologies such as inflammatory and fibrotic diseases and tumor development (Gleizes et al., 1997, Stem Cells 15:190-197). Signaling of the TGFβs is accomplished through binding of the molecules to type I and type II receptors (serine/threonine kinases) on the cell surface. The binding allows receptor II to phosphorylate the receptor I kinase domain, which then propagates the signal through phosphorylation of the Smad proteins (Shi et al., 2003, Cell 113:685-700).

TGFβ1 is synthesized in cells as a 390-amino acid precursor protein composed of a typical leader peptide and a pro-TGFβ1. The precursor protein undergoes a number of intracellular processing steps prior to secretion from the cell. One processing step is the proteolytic digestion of the precursor protein between amino acid residues 278 and 279 by the endopeptidase furin, yielding an N-terminal portion that is the 65-75 kDa latency-associated peptide (LAP), and yielding a C-terminal portion that is the 25-kDa mature TGFβ1 (Blanchette et al., 1997, J. Clin. Invest 99:1974-1983; Dubois et al., 1995, J. Biol. Chem. 270:10618-10624). After the cleavage of the precursor, the mature TGFβ1 is homodimerized and remains noncovalently associated with the LAP until activation (Khalil, 1999, Microbes. Infect. 1:12 55-1263).

LAP, an important component of TGFβ1, is required for efficient secretion of the cytokine, keeps the cytokine in an inactive form, prevents the cytokine from binding to ubiquitous cell surface receptors and maintains the availability of TGFβ1 in a large extracellular reservoir that is readily accessed by activation. Most cells release latent TGFβ1 as a larger complex, in which TGFβ1 is associated with a 120-240-kDa glycoprotein named latent TGFβ1 binding protein (LTBP).

TGFβ1 was initially purified from human platelet with extremely low yield of 40 μg from 800-1000 liters of human blood (Miyazono et al., 1988, J. Biol. Chem. 263:6407-6415). Due to its functional importance, attempts have been made to produce recombinant TGFβ1 in both bacteria and mammalian expression systems. Although attempts to produce TGFβ1 in mammalian cells have been successful, yields have been low (Gentry et al., 1988, Mol. Cell Biol. 8:4162-4168; Gentry et al., 1987, Mol. Cell. Biol. 7:3418-3427; Bourdrel et al., 1993, Protein Expr. Purif. 4:130-140; Archer et al., 1993, Biochemistry 32:1152-1163). Additionally, recombinant systems employed to date have required lengthy purification protocols that further compromised the yield to less than 1-2 mg of recombinant protein per liter of harvest medium.

### SUMMARY

The present invention relates to compositions and methods for the high efficiency production of the transforming growth factor-β (TGFβ) supergene family members.

In one aspect, the invention relates to an isolated mammalian TGFβ-encoding nucleic acid molecule comprising (a) a pro-TGFβpolynucleotide encoding a mammalian pro-TGFβ polypeptide, wherein the pro-TGFβpolynucleotide does not encode a cysteine residue within the first ten amino acid residues of the pro-TGFβpolypeptide; and (b) a signal polynucleotide encoding a heterologous signal polypeptide, wherein the polynucleotide is in frame with the pro-TGFβpolynucleotide.

In another aspect, the invention relates to an isolated mammalian TGFβ-encoding nucleic acid molecule comprising (a) a pro-TGFβpolynucleotide encoding a mammalian pro-TGFβpolypeptide, wherein the pro-TGFβpolynucleotide does not encode a cysteine residue within the first ten amino acid residues of the pro-TGFβpolypeptide; and (b) a signal polynucleotide encoding a heterologous signal polypeptide, wherein the polynucleotide is in frame with the pro TGFβpolynucleotide, and wherein the pro-TGFβpolynucleotide encodes a mammalian pro-TGFβpolypeptide comprising a mature TGFβportion and a LAP portion, and wherein the mature TGFβportion is 95% identical in nucleotide but preferably 100% identical in polypeptide to a mature human TGFβ.

In another aspect, the invention relates to an isolated nucleic acid molecule comprising a pro-TGFβpolynucleotide, a signal polynucleotide, and a tag polynucleotide encoding a purification tag polypeptide, wherein the polynucleotide encoding the purification tag polypeptide is located between, and in frame with, the signal polynucleotide and the polynucleotide encoding the pro-TGFβpolypeptide.

In another aspect, the invention relates to isolated eukaryotic cell lines comprising an isolated mammalian TGFβ-encoding nucleic acid molecule further comprising (a) a pro-TGFβpolynucleotide encoding a mammalian pro-TGFβpolypeptide, wherein the pro-TGFβpolynucleotide does not encode a cysteine residue within the first ten amino acid residues of the pro-TGFβ polypeptide; and (b) a signal polynucleotide encoding a heterologous signal polypeptide, wherein the polynucleotide is in frame with the pro TGFβ polynucleotide.

In another aspect, the invention relates to vectors and expression vectors comprising an isolated mammalian TGFβ-encoding nucleic acid molecule further comprising (a) a pro-TGFβ polynucleotide encoding a mammalian pro-TGFβ polypeptide, wherein the pro-TGFβ polynucleotide does not encode a cysteine residue within the first ten amino acid residues of the pro-TGFβ polypeptide; and (b) a signal polynucleotide encoding a heterologous signal polypeptide, wherein the polynucleotide is in frame with the pro TGFβ polynucleotide. In some aspects, the nucleic acid is operatively linked to the regulatory sequence in an antisense orientation in the expression vector. In other aspects, the nucleic acid is operatively linked to the regulatory sequence in a sense orientation in the expression vector.

In another aspect invention relates to a host cell comprising an isolated mammalian TGFβ-encoding nucleic acid molecule further comprising (a) a pro-TGFβ polynucleotide encoding a mammalian pro-TGFβ polypeptide, wherein the pro-TGFβ polynucleotide does not encode a cysteine residue within the first ten amino acid residues of the pro-TGFβ polypeptide; and (b) a signal polynucleotide encoding a heterologous signal polypeptide, wherein the polynucleotide is in frame with the pro TGFβ polynucleotide. In some aspects, the host cell of is a eukaryote.

In another aspect invention relates to an comprising an isolated mammalian TGFβ-encoding nucleic acid molecule further comprising (a) a pro-TGFβ polynucleotide encoding a mammalian pro-TGFβ polypeptide, wherein the pro-TGFβ polynucleotide does not encode a cysteine residue within the first ten amino acid residues of the pro-TGFβ polypeptide; and (b) a signal polynucleotide encoding a heterologous signal polypeptide, wherein the polynucleotide is in frame with the pro TGFβ polynucleotide.

In another aspect the invention relates to eukaryotic cells containing polynucleotides of the invention.

In another aspect the invention relates to an isolated polypeptide encoded by a nucleic acid further comprising (a) a pro-TGFβ polynucleotide encoding a mammalian pro-TGFβ polypeptide, wherein the pro-TGFβ polynucleotide does not encode a cysteine residue within the first ten amino acid residues of the pro-TGFβ polypeptide; and (b) a signal polynucleotide encoding a heterologous signal polypeptide, wherein the polynucleotide is in frame with the pro TGFβ polynucleotide. In some aspects, the isolated polypeptide has the amino acid sequence of SEQ ID NO:1, SEQ ID NO:2, SEQ ID NO:3, or SEQ ID NO:4. In other aspects, the isolated polypeptide is fused with a heterologous peptide.

In another aspect the invention relates to methods of producing TGFβ polypeptides comprising culturing eukaryotic cells of the invention under conditions wherein greater than 25 mg of mature TGFβ per liter of culture medium is produced, and recovering the TGFβpolypeptide from the isolated cell line or its medium.

In another aspect, the invention relates to a method of producing mature TGFβpolypeptide comprising: (a) culturing a eukaryotic cell line of the invention to produce TGFβ complex in the culture medium, wherein TGFβ complex comprises mature TGFβ polypeptide and LAP polypeptide fused with a purification tag polypeptide; (b) purifying the TGFβ complex by binding the TGFβ complex with a binding agent that specifically binds to the purification tag polypeptide; (c) activating the TGFβ complex to dissociate mature TGFβ polypeptide from associated LAP polypeptide; (d) separating mature TGFβ polypeptide from the LAP polypeptide; and (e) recovering the TGFβ polypeptide from the isolated cell line or its medium.

In another aspect, the invention relates to an isolated Chinese hamster ovary cell line comprising a pro-TGFβ polynucleotide encoding a mammalian pro-TGFβ polypeptide, wherein the polynucleotide does not encode a cysteine residue within the first ten amino acid residues of the pro-TGFβ polypeptide.

In another aspect, the invention relates to a method of producing mature TGFβ polypeptide comprising culturing an isolated eukaryotic cell line comprising a recombinant pro-TGFβ polynucleotide encoding a mammalian pro-TGFβ polypeptide, wherein the polynucleotide does not encode a cysteine residue within the first ten amino acid residues of the pro-TGFβ polypeptide, and wherein the cell line is cultured under conditions that produce greater than 25 mg of mature TGFβ per liter of culture medium, and recovering the TGFβ polypeptide from the isolated cell line or its medium.

### BRIEF DESCRIPTION OF THE DRAWINGS

FIG. 1 shows a schematic representation of the latent TGFβ1 secreted by most cells. TGFβ1 dimer, which is linked noncovalently with LAP dimer, has eight intra- and one inter-subunit disulphide bonds.

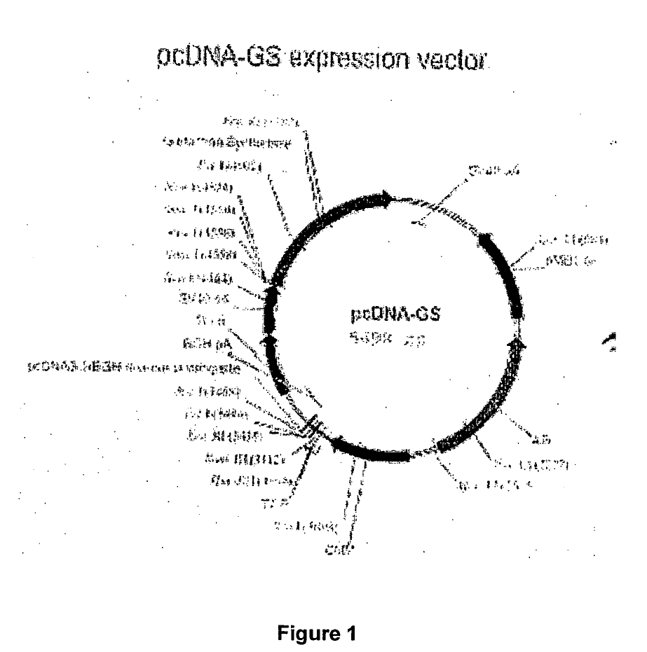

FIG. 2 shows a schematic diagram of the expression vector that was employed for expression of pro-TGFβ1.

FIG. 3 shows an electrophoresis and Western blot analysis of purified TGFβ1. Panel A shows a gel electrophoresis analysis of pro-TGFβ1 purified by a Ni-NTA column. Samples were run on a homogeneous 20% SDS-polyacrylamide gel and the gel was stained with Coomassie blue. Lane 1 shows the results of electrophoresis under nonreducing conditions, and lane 2 shows the results run under reducing conditions. Three major bands showed up which represented pro-TGFβ1, LAP and mature TGFβ1, respectively. Panel B and Panel C show a gel electrophoresis analysis of mature TGFβ after purification by size exclusion chromatography. Samples were run on a homogeneous 20% SDS-polyacrylamide gel. In Panel B, the gel was stained with Coomassie blue and the lanes were as follows: Lane 1, molecular weight markers (kDa); Lane 2, mature TGFβ run under nonreducing conditions; Lane 3, mature TGFβ run under reducing conditions. In Panel C, the gel was silver stained. Lanes 1, 2 and 3 contained 2, 1.5 and 1 μg purified TGFβ1, respectively, run under non-reducing conditions; Lane 4 contained molecular weight markers. Panel D shows a Western blot analysis. A 1.5 μg sample of purified TGFβ1 was run under non-reducing conditions on a 12.5% SDS-polyacrylamide gel. Proteins in the gel were transferred to a PVDF membrane by electroblotting, and TGFβ1 was visualized by immunostaining.

FIG. 4 shows a size exclusion chromatography profile of TGFβ1. A 2 ml sample was loaded onto a HiLoad 16/60 Superdex 200 prep grade column. The equilibration and elution buffer was as follows: 50 mM Glycine, 50 mM sodium chloride, at pH 4.0.

FIG. 5 shows a comparison between purified TGFβ1 and commercial TGFβ1 in binding with human recombinant type II receptor. Samples were made serial dilution from 1000 pg/ml to 31.2 pg/ml. Optical density was measured at wavelength of 450 nm.

FIG. 6 shows BIAcore sensogram analysis of TβRII binding to TGFβ1 immobilized on a CM5 chip using the following concentrations of TβRII: 150, 75, 37.5, 18.75, 9.38, 4.69, 2.34, 1.17, and 0.586 nM.

FIG. 7 shows an analysis of TβRII binding to TGFβ1. Results are shown as a double reciprocal plot of 1/(binding RU) vs. 1/(concentration of TβRII).

FIG. 8 shows a comparison between the biological activity of TGFβ1 and the biological activity of commercial TGFβ1 biological activity. All assays were performed in triplicate and error bars represent the standard error of the mean of the samples.

### DETAILED DESCRIPTION

A. General Overview

The present invention relates to novel compositions and methods for the high efficiency production of the transforming growth factor-beta (TGFβ) supergene family of peptide growth factors. Preferred transforming growth factor-beta supergene family members are TGFβ 1, 2, and 3 in eukaryotic cells. Unexpectedly superior results have been achieved in the production of mammalian TGFβ polypeptides using eukaryotic host cells that have been transformed with a recombinant expression vector containing a gene that encodes a mammalian pro-TGFβ polypeptide. The expression system of the invention has produced a yield of mature TGFβ polypeptide that is at least about ten fold higher than those previously reported.

The TGFβ supergene family consists of a set of growth factors that share at least 25% sequence identity in their mature amino acid sequence with those of TGFβs. Members in this gene family include the transforming growth factors, TGFβ1 through 5; inhibins and activins (inhibin A, inhibin B, activin A and activin AB; bone morphogenic proteins (BMP), such as BMP-2; the decapentaplegic gene complex, DPP-C; Vg1; vgr-1; Müllerian inhibiting substance, MIS; growth differentiation factors, GDF-1 and dorsalin-1, ds1-1. Like TGFβ, they exist as either homo or hetero dimers. See Kingsley, 1994, Genes Dev 8:133-146; Griffith, 1996, Proc Natl. Acad. Sci. U.S.A. 93:878-883; Hogan, 1996, Genes Dev. 10:1580-1594; Massague, 1996, Cell 85:947-950; all of which are incorporated by reference in their entirety for all purposes. Collectively, these TGFβ supergene family members are also referred to herein as “TGFβ”

The five isoforms of TGFβs that have been isolated to date from different species share a 50-80% sequence homology with a conserved Cys 33 in all five sequences. All expressed as disulfide bonded pro-TGFβpeptides. Structurally, the mature form of TGFβ1, 2, and 3 have been shown to be nearly identical. The high sequence and structural homology among the five TGFβ together with their similar expression/activation pathways suggest that the current expression technology be fully applicable for all five TGFβs. (Marks et al., 1991, J. Mol. Biol. 222:581-597, for example.)

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived, from another species or belonging to another antibody class or subclass, as well as fragments of such antibodies, so long as they exhibit the desired biological activity (Cabilly et al., supra; and Morrison et al., 1984, Proc. Natl. Acad. Sci. U.S.A. 81:6851-6855.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., 1986, Nature 321:522-525; Reichmann et al., 1988, Nature 332:323-329; Presta, 1992, Curr. Op. Struct. Biol. 2:593-596. The humanized antibody includes a Primatized™ antibody wherein the antigen-binding region of the antibody is derived from an antibody produced by immunizing macaque monkeys with the antigen of interest.

“Non-immunogenic in a human” means that upon contacting the polypeptide of interest in a physiologically acceptable carrier and in a therapeutically effective amount with the appropriate tissue of a human, no state of sensitivity or resistance to the polypeptide of interest is demonstrable upon the second administration of the polypeptide of interest after an appropriate latent period (e.g., 8 to 14 days).

A “neutralizing antibody” is meant an antibody which is able to block or significantly reduce an effector function of wild type or recombinant TGFB For example, a neutralizing antibody can inhibit or reduce TGFB activation by an agonist antibody, as determined, for example, in a neurite survival assays, a TGFB binding assay, or other assays taught herein or known in the art.

Methods of producing polyclonal and monoclonal antibodies that react specifically with the TGFB proteins are known to those of skill in the art (see, e.g., Coligan, 1991, Current Protocols in Immunology; Harlow et al., supra; Goding, 1986, Monoclonal Antibodies: Principles and Practice (2nd ed.); Kohler et al., 1975, Nature 256:495-497. Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., 1989, Science 246:1275-1281; Ward et al., 1989, Nature 341:544-546.

A number of immunogens comprising portions of TGFB protein can be used to produce antibodies specifically reactive with TGFB protein. For example, recombinant TGFB protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein can also be used either in pure or impure form. The product is then injected into an animal capable of producing antibodies. Either monoclonal or polyclonal antibodies can be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see, Kohler et al., 1976, Eur. J. Immunol. 6:511-519. Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences which encode a monoclonal antibody or a binding fragment thereof by screening a DNA library from human B cells according to the general protocol outlined by Huse et al., 1989, Science, 246:1275-1281.

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-TGFB proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular TGFB ortholog, such as human TGFB, can also be made, by subtracting out other cross-reacting orthologs from a species such as a non-human mammal.

Once the specific antibodies against TGFB protein are available, the protein can be detected by a variety of immunoassay methods. In addition, the antibody can be used therapeutically as TGFB modulators. For a review of immunological and immunoassay procedures, see Stites and Ten, eds., 1991, Basic and Clinical Immunology, 7th ed. Moreover, the immunoassays of the present invention can be performed in any of several configurations, which are reviewed extensively in Maggio, ed., 1980, Enzyme Immunoassay; and Harlow & Lane, supra.

In a further embodiment, antibodies or antibody fragments can be isolated from antibody phage libraries generated using the techniques described in McCafferty et al., 1990, Nature 348:552-554; Clackson et al., 1991, Nature 352:624-628; Marks et al., 1991, J. Mol. Biol. 222:581-597, describe the isolation of murine and human antibodies, respectively, using phage libraries. Subsequent publications describe the production of high affinity (nM range) human antibodies by chain shuffling (Mark et al., 1992, Bio/Technology 10:779-783, as well as combinatorial infection and in vivo recombination as a strategy for constructing very large phage libraries (Waterhouse et al., 1993, Nuc. Acids. Res. 21:2265-2266. Thus, these techniques are viable alternatives to traditional monoclonal antibody hybridoma techniques for isolation of monoclonal antibodies.

The DNA also can be modified, for example, by substituting the coding sequence for human heavy- and light-chain constant domains in place of the homologous murine sequences (Cabilly et al., supra; Morrison, et al., 1984, Proc. Nat. Acad. Sci. U.S.A. 81:6851; or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Chimeric or hybrid antibodies also can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate.

Methods for humanizing non-human antibodies are well known in the art. Generally, a humanized antibody has one or more amino acid residues introduced into it from a source which is nonhuman. These non-human amino acid residues are often referred to as “import” residues, which are typically taken from an “import” variable domain. Humanization can be essentially performed following the method of Winter and co-workers (Jones et al., 1986, Nature 321:522-525; Riechmann et al., 1988, Nature 332:323-322; Verhoeyen et al., 1988, Science 239:1534-1536), by substituting rodent CDRs or CDR sequences for the corresponding sequences of a human antibody. Accordingly, such “humanized” antibodies are chimeric antibodies (Cabilly et al., supra), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., 1993, J. Immunol. 151:2296; Chothia et al., 1987, J. Mol. Biol. 196:901. Another method uses a particular framework derived from the consensus sequence of all human antibodies of a particular subgroup of light or heavy chains. The same framework can be used for several different humanized antibodies (Carter et al., 1992, Proc. Natl. Acad. Sci. U.S.A. 89:4285; Presta et al., 1993, J. Immunol. 151:2623.

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., 1993, Proc. Natl. Acad. Sci. U.S.A. 90:2551; Jakobovits et al., 1993, Nature 362:255-258; Bruggermann et al., 1993, Year in Immuno 7:33. Human antibodies can also be produced in phage-display libraries (Hoogenboom et al., 1991, J. Mol. Biol. 227:381; and Marks et al., 1991, J. Mol. Biol., 222:581).

Bispecific antibodies (BsAbs) are antibodies that have binding specificities for at least two different antigens. BsAbs can be used as tumor targeting or imaging agents and can be used to target enzymes or toxins to a cell possessing the TGFB. Such antibodies can be derived from full length antibodies or antibody fragments (e.g., F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details of generating bispecific antibodies see, for example, Suresh et al., 1986, Methods in Enzymology 121:210.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies can be made using any convenient cross-linking methods. Suitable crosslinking agents are well known in the art, and are disclosed in U.S. Pat. No. 4,676,980, along with a number of cross-linking techniques.

Techniques for generating bispecific antibodies from antibody fragments have also been described in the literature. The following techniques can also be used for the production of bivalent antibody fragments which are not necessarily bispecific. According to these techniques, Fab′-SH fragments can be recovered from E. coli, which can be chemically coupled to form bivalent antibodies. Shalaby et al., 1992, J. Exp. Med. 175:217-225 describe the production of a fully humanized BsAb F(ab′)₂ molecule. Each Fab′ fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also Rodriguez et al., 1992, Int. J. Cancers (Suppl.) 7:45-50.

Various techniques for making and isolating bivalent antibody fragments directly from recombinant cell culture have also been described. For example, bivalent heterodimers have been produced using leucine zippers. Kostelny et al., 1992, J. Immunol. 148:1547-1553. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., 1993, Proc. Natl. Acad. Sci. U.S.A. 90:6444-6448, has provided an alternative mechanism for making BsAb fragments. The fragments comprise a heavy-chain variable domain (VH) connected to a light-chain variable domain (V_{L}) by a linker which is too short to allow pairing between the two domains on the same chain. Accordingly, the V_{H} and V_{L} domains of one fragment are forced to pair with the complementary V_{H} and V_{L} domains of another fragment, thereby forming two antigen-binding sites. Another strategy for making BsAb fragments by the use of single-chain Fv (sFv) dimers has also been reported. See Gruber et al., 1994, J. Immunol. 152:5368.

In one aspect, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

“Increased or enhanced expression or activity of a polypeptide of the present invention,” or “increased or enhanced expression or activity of a polynucleotide encoding a polypeptide of the present invention,” refers to an augmented change in activity of the polypeptide or protein. Examples of such increased activity or expression include the following: Activity of the protein or expression of the gene encoding the protein is increased above the level of that in wild-type, non-transgenic controls. Activity of the protein or expression of the gene encoding the protein is in an organ, tissue or cell where it is not normally detected in wild-type, non-transgenic controls (i.e., spatial distribution of the protein or expression of the gene encoding the protein is altered). Activity of the protein or expression of the gene encoding the protein is increased when activity of the protein or expression of the gene encoding the protein is present in an organ, tissue or cell for a longer period than in a wild-type, non-transgenic controls (i.e., duration of activity of the protein or expression of the gene encoding the protein is increased).

“Decreased expression or activity of a protein or polypeptide of the present invention,” or “decreased expression or activity of a nucleic acid or polynucleotide encoding a protein of the present invention,” refers to a decrease in activity of the protein. Examples of such decreased activity or expression include the following: Activity of the protein or expression of the gene encoding the protein is decreased below the level of that in wild-type, non-transgenic controls.

The term “sequence identity” refers to a measure of similarity between amino acid or nucleotide sequences, and can be measured using methods known in the art, such as those described in further detail below.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region (see, e.g., SEQ ID NO:1), when compared and aligned for maximum correspondence over a comparison window, or designated region as measured using one of the following sequence comparison algorithms or by manual alignment and visual inspection.

The phrase “substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the sequence comparison algorithms discussed in detail below or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

The phrase “sequence similarity” in the context of two nucleic acids or polypeptides, refers to two or more sequences that are identical or in the case of amino acids, have homologous amino acid substitutions at either 50%, often at least 60%, often at least 70%, preferably at least 80%, most preferably at least 90% or at least 95% of the indicated positions.

“Control sequences” or “regulatory sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

“Vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “promoter” refers to a region or sequence determinants located upstream or downstream from the start of transcription and which are involved in recognition and binding of RNA polymerase and other proteins to initiate transcription.

“Cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny cannot be precisely identical in DNA content, due to deliberate or inadvertent mutations. Mutant progeny that have the same function or biological activity as screened for in the originally transformed cell are included. Where distinct designations are intended, it will be clear from the context.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny can not, in fact, be identical to the parent cell, but are still included within the scope of the term “host cell” as used herein.

An “expression cassette” refers to a nucleic acid construct, which when introduced into a host cell, results in transcription and/or translation of a RNA or polypeptide, respectively. Expression cassettes can be derived from a variety of sources depending on the host cell to be used for expression. For example, an expression cassette can contain components derived from a viral, bacterial, insect, or mammalian source. In the case of both expression of transgenes and inhibition of endogenous genes (e.g., by antisense, or sense suppression) one of skill will recognize that the inserted polynucleotide sequence need not be identical and can be “substantially identical” to a sequence of the gene from which it was derived. As explained below, these variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “polynucleotide sequence from” a particular gene. In addition, the term specifically includes sequences (e.g., full length sequences) substantially identical with a gene sequence encoding a polypeptide of the present invention, e.g., SEQ ID NO:1, SEQ ID NO:2, SEQ ID NO:3 and SEQ ID NO:4, and that encode proteins that retain the function of a protein of the present invention, e.g., specific binding to their respective TGFβ supergene family members.

In the case of polynucleotides used to inhibit expression of an endogenous gene, the introduced sequence need not be perfectly identical to a sequence of the target endogenous gene. The introduced polynucleotide sequence will typically be at least substantially identical to the target endogenous sequence.

“Receptor” denotes a cell-associated protein, for example a TGFβ receptor (TGFβ-R; type I or -type II, both of which are serine/threonine kinases) on the cell surface, that binds to a bioactive molecule termed a “ligand,” for example, a TGFβ dimer. This interaction mediates the effect of the ligand on the cell. Receptors can be membrane bound, cytosolic or nuclear; monomeric (e.g., thyroid stimulating hormone receptor, beta-adrenergic receptor) or multimeric. Membrane-bound receptors, for example TGFβ-R, are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

“High affinity” for a ligand refers to an equilibrium association constant (Ka) of at least about 10³M⁻¹, at least about 10⁴M⁻¹, at least about 10⁵M⁻¹, at least about 10⁶M⁻¹, at least about 10⁷M⁻¹, at least about 10⁸M⁻¹, at least about 10⁹M¹, at least about 10¹° M⁻¹, at least about 10¹¹M⁻¹, or at least about 10¹²M¹ or greater, e.g., up to 10¹³M⁻¹ or 10¹⁴M⁻¹ or greater. However, “high affinity” binding can vary for other ligands.

“Kₐ”, as used herein, is intended to refer to the equilibrium association constant of a particular ligand-receptor interaction, e.g., antibody-antigen interaction. This constant has units of 1/M.

“K_{d}”, as used herein, is intended to refer to the equilibrium dissociation constant of a particular ligand-receptor interaction. This constant has units of M.

“kₐ”, as used herein, is intended to refer to the kinetic association constant of a particular ligand-receptor interaction. This constant has units of 1/Ms.

“k_{d}”, as used herein, is intended to refer to the kinetic dissociation constant of a particular ligand-receptor interaction. This constant has units of 1/s.

The phrase “specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

The phrase “specifically bind(s)” or “bind(s) specifically” when referring to a peptide refers to a peptide molecule which has intermediate or high binding affinity, exclusively or predominately, to a target molecule. The phrase “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically react with TGFβ domain-containing proteins. Typically a specific or selective reaction will be at least twice background signal or noise and more typically more than 10 times background. Specific binding between TGFβ and TGFβ-R means a binding affinity of at least 10³ M⁻¹, and preferably 10⁵, 10⁶, 10⁷, 10⁸, 10⁹ or 10¹⁰ M⁻¹. The binding affinity of TGFβ and TGFβ-R is preferably between about 10⁶ M⁻¹ to about 10¹° M⁻¹.

“Particular ligand-receptor interactions” refers to the experimental conditions under which the equilibrium and kinetic constants are measured.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compositions of the invention can be administered.

“Concomitant administration” of a known cancer therapeutic drug with a pharmaceutical composition of the present invention means administration of the drug and the collectin and/or surfactant protein composition at such time that both the known drug and the composition of the present invention will have a therapeutic effect. Such concomitant administration may involve concurrent (i.e., at the same time), prior, or subsequent administration of the antimicrobial drug with respect to the administration of a compound of the present invention. A person of ordinary skill in the art would have no difficulty determining the appropriate timing, sequence and dosages of administration for particular drugs and compositions of the present invention.

“Treating” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination. Accordingly, the term “treating” includes the administration of the compounds or agents of the present invention to inhibit a disease. Accordingly, the term “treating” includes the administration of the compounds or agents of the present invention to prevent or delay, to alleviate, or to arrest or inhibit development of the symptoms or conditions associated with a disease or other disorder. The term “therapeutic effect” refers to the reduction, elimination, or prevention of the disease, symptoms of the disease, or side effects of the disease in the subject.

Additional fusion proteins of the invention can be generated through the techniques of gene-shuffling, motif-shuffling, exon-shuffling, or codon-shuffling (collectively referred to as “DNA shuffling”). DNA shuffling can be employed to modulate the activities of polypeptides of the present invention thereby effectively generating agonists and antagonists of the polypeptides. See, for example, U.S. Pat. Nos. 5,605,793; 5,811,238; 5,834,252; 5,837,458; Patten et al., 1997, Curr. Opinion Biotechnol. 8:724-733; Harayama, 1998, Trends Biotechnol. 16:76-82; Hansson et al., 1999, J. Mol. Biol. 287:265-276; Lorenzo et al., 1998, Biotechniques 24:308-313. (Each of these documents is hereby incorporated by reference). In one embodiment, one or more components, motifs, sections, parts, domains, fragments, and the like, of coding polynucleotides of the invention, or the polypeptides encoded thereby can be recombined with one or more components, motifs, sections, parts, domains, fragments, and the like, of one or more heterologous molecules.

The term “epitope-tagged” when used herein refers to a chimeric polypeptide comprising TGFβ fused to a “tag polypeptide”. The tag polypeptide has enough residues to provide an epitope against which an antibody can be made, yet is short enough such that it does not interfere with biological activity of the TGFβ. The tag polypeptide preferably also is fairly unique so that the antibody does not substantially cross-react with other epitopes. Suitable tag polypeptides generally have at least six amino acid residues and usually between about 8-50 amino acid residues (preferably between about 9-30 residues). Preferred are poly-histidine sequences, which bind nickel, allowing isolation of the tagged protein by Ni-NTA chromatography as described for example in Lindsay, et al. Neuron 17:571-574, 1996.

The nucleic acids of the invention are present in whole cells, in a cell lysate, or in a partially purified or substantially pure form. A nucleic acid is “isolated” when purified away from other cellular components or other contaminants, e.g., other cellular nucleic acids or proteins, by standard techniques, including alkaline/SDS treatment, CsCl banding, column chromatography, agarose gel electrophoresis and others well known in the art (See, e.g., Sambrook, Tijssen and Ausubel discussed herein and incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect or mammalian systems. Alternatively, these nucleic acids can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, Tijssen and Ausubel. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

“Essentially pure” protein means a composition comprising at least about 90% by weight of the protein, based on total weight of the composition, preferably at least about 95% by weight. “Essentially homogeneous” protein means a composition comprising at least about 99% by weight of protein, based on total weight of the composition.

“Inhibitors,” “activators,” and “modulators” of TGFβ activity are used to refer to inhibitory, activating, or modulating molecules, respectively, identified using in vitro and in vivo assays for TGFβ binding or signaling, e.g., ligands, agonists, antagonists, and their homologs and mimetics.

The term “modulator” includes inhibitors and activators. Inhibitors are agents that, e.g., bind to, partially or totally block stimulation, decrease, prevent, delay activation, inactivate, desensitize, or down regulate the activity of TGFβs, e.g., antagonists. Activators are agents that, e.g., bind to, stimulate, increase, open, activate, facilitate, enhance activation, sensitize or up regulate the activity of TGFβs, e.g., agonists. Modulators include agents that, e.g., alter the interaction of TGFβs with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring TGFβ ligands, e.g., with altered activity, as well as naturally occurring and synthetic ligands, antagonists, agonists, small chemical molecules and the like. Such assays for inhibitors and activators include, e.g., applying putative modulator compounds to a cell expressing a TGFβ and then determining the functional effects on TGFβ signaling. Samples or assays comprising TGFβ that are treated with a potential activator, inhibitor, or modulator are compared to control samples without the inhibitor, activator, or modulator to examine the'extent of inhibition. Control samples (untreated with inhibitors) can be assigned a relative TGFβ activity value of 100%. Inhibition of TGFβ is achieved, for example, when the TGFβ activity value relative to the control is about 80%, optionally 50% or 25-0%. Activation of TGFβ is achieved when the TGFβ activity value relative to the control is 110%, optionally 150%, optionally 200-500%, or 1000-3000% higher.

This invention relies on routine techniques in the field of recombinant genetics. Basic texts disclosing the general methods of use in this invention include Sambrook et al., Molecular Cloning, A Laboratory Manual, 2nd ed., 1989; Kriegler, Gene Transfer and Expression: A Laboratory Manual, 1990; and Ausubel et al., eds., Current Protocols in Molecular Biology, 1994, all of which are incorporated by reference in their entireties for all purposes.

B. Pro-TGFβ Polynucleotides

As used herein, a “pro-TGFβ polynucleotide” refers to a polynucleotide that encodes a mammalian pro-TGFβ polypeptide. Polynucleotides of the invention can be derived from natural sources or they can be synthetically produced. The pro-TGFβ polynucleotide can encode any mammalian pro-TGFβ1, pro-TGFβ2, or pro-TGFβ polypeptide. A mammalian pro-TGFβ polypeptide typically contains a latency associated peptide (LAP) portion and a mature TGFβ portion. Preferred pro-TGFβ polynucleotides of the invention are those that encode a pro-TGFβ having a mature TGFβ portion that is at least 90% identical to a mature mammalian TGFβ polypeptide, and more preferably, at least 95% identical to a mature mammalian TGFβ polypeptide.

The pro-TGFβ polypeptide sequence, and the associated nucleic acid sequence, for any particular species can be readily ascertained by techniques that are well known in the art. The polypeptide sequences for several mammalian TGFβ polypeptides, both the mature polypeptide and the LAP polypeptide, can be found at the National Center for Biotechnology Information (NCBI) web site for the following organisms: TGFβ1-human (NCBI Accession No: P01137); TGFβ1-sheep (NCBI Accession No: P50414); TGFβ1-rat (NCBI Accession No: P17246); TGFβ1-pig (NCBI Accession No: P07200); TGFβ1-bovine (NCBI Accession No: P18341); TGFβ1-domestic guinea pig (NCBI Accession No: □9Z1T6); TGFβ1-horse (NCBI Accession No: O19011); TGFβ1-dog (NCBI Accession No: P54831); TGFβ1-mouse (NCBI Accession No: PO4202); TGFβ1-Aftrican Green Monkey (NCBI Accession No: P09533); TGFβ2-human (NCBI Accession No: P08112); TGFβ2-rat (NCBI Accession No: NP 112393); TGFβ3-human (NCBI Accession No: P10600).

Preferred pro-TGFβ polynucleotides of the invention are those that encode a polypeptide having a mature TGFβ portion that is at least 90% identical to mature human TGFβ1 (SEQ ID NO:1; residues 279-390), mature human TGFβ2 (SEQ ID NO:3; residues 303-404), and mature human TGFβ3 (SEQ ID NO:4; residues 301-402), and preferably at least 95% identical to a mature human TGFβ1, 2, or 3, and more preferably at least 98% identical to mature human TGFβ1, 2, or 3.

Preferred pro-TGFβ polynucleotides of the invention are those that encode a pro-TGFβ polypeptide having a LAP portion that this at least 90% percent identical to a the LAP portion of a mammalian TGFβ polypeptide, and more preferably, at least 95% identical to the LAP portion of a mammalian TGFβ polypeptide. Particularly preferred TGFβpolynucleotides of the invention are those that encode a polypeptide having a LAP portion that is at least 90% identical to the LAP portion of human TGFβ1 (SEQ ID NO:1; residues 30-278), the LAP portion of human TGFβ2 (SEQ ID NO:2; residues 20-302), the LAP portion of human TGFβ3 (SEQ ID NO:4; residues 21-300), or the LAP portion of porcine TGFβ1 (SEQ ID NO:2; residues 30-278), and more preferably, at least 95% identical to the LAP portion of human TGFβ1, human TGFβ2, human TGFβ3, or porcine TGFβ1.

Preferred pro-TGFβ polynucleotides of the invention are those that encode a mammalian pro-TGFβ polypeptide having a LAP portion that does not contain a cysteine residue within the first 10 amino acids. More preferred TGFβ polynucleotides of the invention are those that encode a polypeptide having a LAP portion as follows: (i) a LAP portion of human TGFβ1 (SEQ ID NO:1; residues 30-278), wherein the cysteine at residue 33 has been substituted by another amino acid; (ii) a LAP portion of human TGFβ2 (SEQ ID NO:2; residues 20-302), wherein the cysteine at residue 24 has been substituted by another amino acid; (iii) a LAP portion of human TGFβ3 (SEQ ID NO:4; residues 21-300), wherein the cysteine at residue 27 has been substituted by another amino acid; (iv) a LAP portion of porcine TGFβ1 (SEQ ID NO:2; residues 30-278), wherein the cysteine at residue 33 has been substituted by another amino

In a preferred embodiment, the indicated cysteine residue in the LAP polypeptide sequences discussed above is replaced with an amino acid selected from the group consisting of: Ala, Gly, Thr, Asp, Asn, Glu, Gln, Val, Tyr, Ile, Leu, Met, Lys, His, Trp, Phe, Arg, Pro. In a particularly preferred embodiment of the invention, the indicated cysteine residue in the LAP polypeptide sequences discussed above is replaced with a serine residue.

| TGFβ1 Precursor-human(NCBI Accession No: P01137)(the LAP portion is | |
|---|---|
| shown underlined): | |
| (SEQ ID NO: 1) | |
| 1 | mppsglrlll lllpllwllv ltpgrpaagl stcktidmel vkrkrieair gqilsklrla |
| 61 | sppsqgevpp gplpeavlal ynstrdrvag esaepepepe adyyakevtr vlmvethnei |
| 121 | ydkfkqsths iymffntsel reavpepvll sraelrllrl klkveqhvel yqkysnnswr |
| 181 | ylsnrllaps dspewlsfdv tgvvrqwlsr ggeiegfrls ahcscdsrdn tlqvdingft |
| 241 | tgrrgdlati hgmnrpflll matpleraqh lqssrhrral dtnycfsste knccvrqlyi |
| 301 | dfrkdlgwkw ihepkgyhan fclgpcpyiw sldtqyskvl alynqhnpga saapccvpqa |
| 361 | leplpivyyv grkpkveqls nmivrsckcs |
| TGFβ1 Precursor-porcine(NCBI Accession No: P07200)(the LAP portion | |
| is shown underlined): | |
| (SEQ ID NO: 2) | |
| 1 | mppsglrllp lllpllwllv ltpgrpaagl stcktidmel vkrkrieair gqilsklrla |
| 61 | sppsqgdvpp gplpeavlal ynstrdrvaq esvepepepe adyyakevtr vlmlesgnqi |
| 121 | ydkfkgtphs lymlfntsel reavpepvll sraelrllrl klkveqhvel yqkysndswr |
| 181 | ylsnrllaps dspewlsfdv tgvvrqwltr reaiegfrls ahcscdskdn tlhveingfn |
| 241 | sgrrgdlati hgmnrpflll matpleraqh lhssrhrral dtnycfsste knccvrqlyi |
| 301 | dfrkdlgwkw ihepkgyhan fclgpcpyiw sldtqyskvl alynqhnpga saapccvpqa |
| 361 | leplpivyyv grkpkveqls nmivrsckcs |
| TGFβ2 Precursor-Human(NCBI Accession No: P08112)(the LAP portion | |
| is shown underlined): | |
| (SEQ ID NO: 3) | |
| 1 | mhycvlsafl ilhlvtvals lstcstldmd qfmrkrieai rgqilsklkl tsppedypep |
| 61 | eevppevisi ynstrdllqe kasrraaace rersdeeyya kevykidmpp ffpsenaipp |
| 121 | tfyrpyfriv rfdvsamekn asnlvkaefr vfrlqnpkar vpeqrielyq ilkskdltsp |
| 181 | tqryidskvv ktraegewls fdvtdavhew lhhkdrnlgf kislhcpcct fvpsnnyiip |
| 241 | nkseelearf agidgtstyt sgdqktikst rkknsgktph lllmllpsyr lesqqtnrrk |
| 301 | kraldaaycf rnvqdncclr plyidfkrdl gwkwihepkg ynanfcagac pylwssdtqh |
| 361 | srvlslynti npeasaspcc vsqdleplti lyyigktpki eqlsnmivks ckcs |
| TGFβ3 Precursor-Human(NCBI Accession No: P10600)(the LAP portion | |
| is shown underlined): | |
| (SEQ ID NO: 4) | |
| 1 | mkmhlqralv vlallnfatv slslstcttl dfghikkkrv eairgqilsk lrltsppept |
| 61 | vmthvpyqvl alynstrell eemhgereeg ctqentesey yakeihkfdm iqglaehnel |
| 121 | avcpkgitsk vfrfnvssve knrtnlfrae frvlrvpnps skrneqriel fqilrpdehi |
| 181 | akqryiggkn lptrgtaewl sfdvtdtvre wllrresnlg leisihcpch tfqpngdile |
| 241 | nihevmeikf kgvdneddhg rgdlgrlkkq kdhhnphlil mmipphrldn pgqggqrkkr |
| 301 | aldtnycfrn leenccvrpl yidfrqdlgw kwvhepkgyy anfcsgpcpy lrsadtthst |
| 361 | vlglyntlnp easaspccvp qdlepltily yvgrtpkveq lsnmvvksck cs |

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence can include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence can be inserted, deleted, or substituted with another amino acid. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for example, the amino acid sequence of any portion of SEQ ID NO:1, SEQ ID NO:2, SEQ ID NO:3., or SEQ ID NO:4, can be determined conventionally using known computer programs.

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity is evaluated using any of the variety of sequence comparison algorithms and programs known in the art. Such algorithms and programs include, but are by no means limited to, TBLASTN, BLASTP, FASTA, TFASTA, CLUSTALW, FASTDB, the disclosures of which are incorporated by reference in their entireties. Pearson et al., 1988, Proc. Natl. Acad. Sci. U.S.A. 85:2444-2448; Altschul et al., 1990, J. Mol. Biol. 215:403410; Thompson et al., 1994, Nucleic Acids Res. 22:4673-4680; Higgins et al., 1996, Meth. Enzymol. 266:383402; Altschul et al., 1993, Nature Genetics 3:266-272; and Brutlag et al., 1990, Comp. App. Biosci. 6:237-24.

A preferred method for determining the best overall match between a query sequence (a sequence of the present invention) and a subject sequence, also referred to as a global sequence alignment, can be determined using the FASTDB computer program based on the algorithm of Brutlag et al., 1990, Comp. App. Biosci. 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence that are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to be made for the purposes of the present invention.

Another preferred method is using the BLAST programs. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix, the disclosures of which are incorporated by reference in their entireties). Less preferably, the PAM or PAM250 matrices can also be used (see, e.g., Schwartz, et al., eds., 1978, Matrices For Detecting Distance Relationships: Atlas Of Protein Sequence And Structure, Washington: National Biomedical Research Foundation, the disclosure of which is incorporated by reference in its entirety). The BLAST programs evaluate the statistical significance of all high-scoring segment pairs identified, and preferably select those segments which satisfy a user-specified threshold of significance, such as a user-specified percent homology. Preferably, the statistical significance of a high-scoring segment pair is evaluated using the statistical significance formula of Karlin, the disclosure of which is incorporated by reference in its entirety. The BLAST programs can be used with the default parameters or with modified parameters provided by the user. Gonnet et al., 1992, Science 256:1443-1445; Henikoff et al., 1993, Proteins 17:49-61; Karlin et al., 1990.

C. Pro-TGFβ Polynucleotides Uses

Isolation of Nucleic Acids Encoding Pro-TGFβ Polynucleotide Family Members

Pro-TGFβ polynucleotides, polymorphic variants, orthologs, and alleles that are substantially identical to sequences provided herein, as well as other TGFβ supergene family members, can be isolated using TGFβ nucleic acid probes and oligonucleotides under stringent hybridization conditions, by screening libraries. Alternatively, expression libraries can be used to clone TGFβ protein, polymorphic variants, orthologs, and alleles by detecting expressed homologs immunologically with antisera or purified antibodies made against human TGFβ or portions thereof.

To make a cDNA library, one should choose a source that is rich in TGFβ RNA. The mRNA is then made into cDNA using reverse transcriptase, ligated into a recombinant vector, and transfected into a recombinant host for propagation, screening and cloning. Methods for making and screening cDNA libraries are well known (see, e.g., Gubler et al., 1983, Gene 25:263-269; Sambrook et al., 1983, supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton et al., 1977, Science 196:180-182. Colony hybridization is carried out as generally described in Grunstein et al., 1975, Proc. Natl. Acad. Sci. U.S.A. 72:3961-3965.

One exemplary method of isolating pro-TGFβ nucleic acid and its orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; Innis et al., eds., 1990, PCR Protocols: A Guide to Methods and Applications. Methods such as polymerase chain reaction (PCR) and ligase chain reaction (LCR) can be used to amplify nucleic acid sequences of human pro-TGFβ directly from mRNA, from cDNA, from genomic libraries or cDNA libraries. Degenerate oligonucleotides can be designed to amplify TGFβ homologs using the sequences provided herein. Restriction endonuclease sites can be incorporated into the primers. Polymerase chain reaction or other in vitro amplification methods can also be useful, for example, to clone nucleic acid sequences that code for proteins to be expressed, to make nucleic acids to use as probes for detecting the presence of TGFβ encoding mRNA in physiological samples, for nucleic acid sequencing, or for other purposes. Genes amplified by the PCR reaction can be purified from agarose gels and cloned into an appropriate vector.

Gene expression of TGFβ can also be analyzed by, techniques known in the art, e.g., reverse transcription and amplification of mRNA, isolation of total RNA or poly A⁺ RNA, northern blotting, dot blotting, in situ hybridization, RNase protection, high density polynucleotide array technology, e.g., and the like.

Nucleic acids encoding TGFβ or pro-TGFβ protein can be used with high density oligonucleotide array technology (e.g., GeneChip™) to identify TGFβ and pro-TGFβ protein, orthologs, alleles, conservatively modified variants, and polymorphic variants in this invention. In the case where the homologs being identified are linked to TGFβ related diseases, they can be used with GeneChip™ as a diagnostic tool in detecting the disease in a biological sample, see, e.g., Gunthand et al., 1998, AIDS Res. Hum. Retroviruses 14:869-876; Kozal et al., 1996, Nat. Med. 2:753-759; Matson et al., 1995, Anal. Biochem. 224:110-106; Lockhart et al., 1996, Nat. Biotechnol: 14:1675-1680; Gingeras et al., 1998, Genome Res. 8:435-448; Hacia, et al., 1998, Nucleic Acids Res. 26:3865-3866.

Expression of Pro-TGFβ Polynucleotide Family Members in Prokaryotes and Eukaryotes

To obtain high level expression of a cloned gene, such as those cDNAs encoding EDG, one typically subclones EDG into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al., supra. Bacterial expression systems for expressing the EDG protein are available in, e.g., E. coli, Bacillus sp., and Salmonella (Palva et al., 1983, Gene 22:229-235; Mosbach et al., 1983, Nature 302:543-545. Kits for such expression systems are commercially available. Eukaryotic expression systems for mammalian cells, yeast, and insect cells are well known in the art and are also commercially available. In one preferred embodiment, retroviral expression systems are used in the present invention.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the EDG encoding nucleic acid in host cells. A typical expression cassette thus contains a promoter operably linked to the nucleic acid sequence encoding EDG and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc. Sequence tags may be included in an expression cassette for nucleic acid rescue. Markers such as fluorescent proteins, green or red fluorescent protein, β-gal, CAT, and the like can be included in the vectors as markers for vector transduction.

Expression vectors containing regulatory elements from eukaryotic viruses are typically used in eukaryotic expression vectors, e.g., SV40 vectors, papilloma virus vectors, retroviral vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal.

In one embodiment, the vectors of the invention have a regulatable promoter, e.g., tet-regulated systems and the RU-486 system (see, e.g., Gossen and Bujard, 1992, Proc. Nat'l Acad. Sci. USA 89:5547; Oligino et al., 1998, Gene Ther. 5:491-496; Wang et al., 1997, Gene Ther. 4:432-441; Neering et al., 1996, Blood 88:1147-1155; and Rendahl et al., 1998, Nat. Biotechnol. 16:757-761. These impart small molecule control on the expression of the candidate target nucleic acids. This beneficial feature can be used to determine that a desired phenotype is caused by a transfected cDNA rather than a somatic mutation.

Some expression systems have markers that provide gene amplification such as thymidine kinase and dihydrofolate reductase. Alternatively, high yield expression systems not involving gene amplification are also suitable, such as using a baculovirus vector in insect cells, with a EDG encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

The use of pro-TGFβ polynucleotides of the invention to achieve the high efficiency expression of mammalian pro-TGFβ polypeptides in eukaryotic cells is contemplated. Preferred host cells for expression of the TGFβ constructs of the invention are derived from multicellular organisms. Examples of invertebrate cells include insect cells such as Drosophila S2 and Spodoptera Sf9 or Spodoptera High 5 cells, as well as plant cells. Examples of useful mammalian host cell lines include Chinese hamster ovary (CHO), NS0, NS1 and COS cells. More specific examples include the following monkey kidney CV1 line transformed by SV40 (COS-7, ATCC CRL 1651); human embryonic kidney line 293 or 293 cells subcloned for growth in suspension culture (Graham et al., 1977, J. Gen Virol. 36:59); Chinese hamster ovary cells/−DHFR (Urlaub and Chasin, 1980, Proc. Natl. Acad. Sci. USA 77:4216); mouse sertoli cells (TM4) (Mather, 1980, Biol. Reprod. 23:243-251); human lung cells (W138, ATCC CCL 75); human liver cells (Hep G2, HB 8065); and mouse mammary tumor cells (MMT 060562, ATCC CCL51). In a preferred embodiment of the invention, the use of CHO cells is contemplated.

Other eukaryotic organisms that can be employed to achieve expression of the TGFβ polynucleotides of the invention are eukaryotic microbes such as filamentous fungi or yeast. Saccharomyces cerevisiae is a commonly used lower eukaryotic host microorganism. Others include Schizosaccharomyces ponzbe (Beach and Nurse, 1981, Nature 290:140; EP 139,383 published Can 2, 1985); Kluyveromyces hosts (U.S. Pat. No. 4,943,529; Fleer et a, 1991, Bio/Technology 9:968-975 such as, e.g., K. lactis (MW98-8C, CBS683, CBS4574; Louvencourt et al., 1983, J. Bacteriol. 154(2):737-742; K. fragilis (ATCC 12,424), K. bulgaricus (ATCC 16,045), K. wickeramii (ATCC 24,178), K. waitii (ATCC 56,500), K. drosophilarum (ATCC 36,906; Van den Berg et al., 1990, Bio/Technology 8:135), K. thermotolerans, and K. marxianus; yarrowia (EP 402,226); Pichia pastoris (EP 183,070; Sreekrishna et al., 1988, J. Basic Microbiol., 28:265-278); Candida; Trichoderma reesia (EP 244,234); Neurospora crassa (Case et al., 1979, Proc. Natl. Acad. Sci. USA 76:5259-5263); Schwanniomyces such as Schwanniomyces occidentalis (EP 394,538 published Oct. 31, 1990); and filamentous fungi such as, e.g., Neurospora, Penicillium, Tolypocladium (WO 91/00357 published 1991), and Aspergillus hosts such as A. nidulans (Ballance et al., 1983, Biochem. Biophys. Res. Conzmun. 112:284-289; Tilburn et al., 1983, Gene 26:205-221; Yelton et al., 1984, Proc. Natl. Acad. Sci. USA 81:1470-1474; and A. niger (Kelly and Hynes, 1985, EMBO J., 4:475479). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts can be found in Anthony, 1982, The Biochemistry of Methylotrophs, 269.

The selection of an appropriate host cell can be made by one of skill in the art. Host cells are transfected or transformed with nucleic acid molecules of the invention and cultured in conventional nutrient media that is modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of mammalian cell cultures can be found in M. Butler, ed., 1991, Mammalian Cell Biotechnology: a Practical Approach, IRL Press.

Methods of eukaryotic cell transfection or transformation are known to the ordinarily skilled artisan. For eukaryotic cells without cell walls, the calcium phosphate precipitation method of Graham and van der Eb, 1978, Virology 52:456457 can be employed. Other techniques that can be employed include the use of liposomes, electroporation, microinjection, cell fusion, and DEAE-dextran. Various techniques for transforming mammalian cells are described in Keown et al., 1990, Methods in Enzymology 185:527-537) and Mansour et al., 1988, Nature 336:348-352. General aspects of mammalian cell host system transfections have been described in U.S. Pat. No. 4,399,216. Infection with Agrobacterium tumefaciens can be used for the transformation of certain plant cells, as described by Shaw et al., 1983, Gene 23:315) and in WO 89/05859 (published Jun. 29, 1989). Transformations into yeast are typically carried out according to the method of Van Solingen et al., 1977, J. Bact. 130:946); and Hsiao et al., 1979, Proc. Natl. Acad. Sci. (USA) 76:3829.

The TGFβ encoding nucleic acid molecules of the invention will typically be inserted into a replicable vector for cloning (amplification of the DNA) or for expression. Various vectors are publicly available. The vector can, for example, be in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components can include, but are not limited to, one or more of an origin of replication, marker gene(s), enhancer element(s), promoter(s), and transcription termination sequence(s). Construction of suitable vectors containing one or more of these components employs standard techniques that are known to the skilled artisan.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ, plasmid origin is suitable for yeast, and various viral origins (SV40, polyoma, adenovirus, VSV or BPV) are useful for cloning vectors in mammalian cells. Expression and cloning vectors will typically contain a selection gene, also termed a selectable marker.

Suitable selectable markers for mammalian cells enable the identification of transfected cells. Examples of suitable marker systems include the dihydrofolate reductase (DHFR)/methotrexate system and the glutamine synthetase/methionine sulfoxamine system that are well known in the art. These systems are particularly applicable for use with Chinese hamster ovary cells (Goeddel, 1990, Methods in Enzymology 185:543-551). Selectable markers can also be used to increase the productivity of a recombinant cell line by gene amplification of the inserted gene. Thymidine kinase is another selectable marker that can be used in mammalian cells. A suitable selection gene for use in yeast is the trp1 gene present in the yeast plasmid YRp7 (Stinchcomb et al., 1979, Nature 282:39; Kingsman et al., 1979, Gene 7:141; Tschemper et al., 1980, Gene 10:157. The tip1 gene provides a selection marker for a mutant strain of yeast lacking the ability to grow in tryptophan, for example, ATCC No. 44076 or PEP4-1 (Jones, 1977, Genetics 85:12).

Expression and cloning vectors usually contain a promoter operably linked to the TGFβ-encoding nucleic acid sequence to direct mRNA synthesis. Promoters recognized by a variety of potential host cells are well known. Promoter useful in mammalian cells include, for example, the following: (i) promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40); heterologous mammalian promoters including, for example, the actin promoter, an immunoglobulin promoter, and promoters derived from heat-shock genes.

Examples of suitable promoting sequences for use with yeast hosts include the promoters for 3-phosphoglycerate kinase (Hitzeman et al., 1980, J. Biol. Chem. 255:2073) or other glycolytic enzymes (Hess et al., 1968, J. Adv. Enzyme Reg. 7:149; Holland, 1978, Biochemistry 17:4900), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Transcription of a TGFβ-encoding nucleic acid by higher eukaryotes can be increased by inserting an enhancer sequence into the vector. Enhancers are cis-acting elements of DNA, usually about from 10 to 300 bp, which act on a promoter to increase its transcription. Many enhancer sequences are now known from mammalian genes, e.g., globin, elastase, albumin, alpha-fetoprotein, and insulin. Typically, however, an enhancer from a eukaryotic cell virus will be employed. Examples include the SV40 enhancer on the late side of the replication origin (bp 100-270), the cytomegalovirus early promoter enhancer, the polyoma enhancer on the late side of the replication origin, and adenovirus enhancers. The enhancer can be spliced into the vector at a position 5′ or 3′ to the TGFβ coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences include, for example, nucleotide segments transcribed as polyadenylated fragments in the untranslated portion of the mRNA encoding a TGFβ polypeptide. Such sequences can be readily obtained from eukaryotic or viral genes.

Nucleic acid molecules of the invention can contain a polynucleotide that encodes a natural signal polypeptide or a heterologous signal polypeptide to facilitate secretion of TGFβ polypeptides into the culture medium. A “heterologous signal polypeptide” refers to any polypeptide sequence that facilitates secretion of TGFβ polypeptides into the culture medium, other than the signal polypeptide that is associated with a particular TGFβ in nature. In a preferred embodiment, nucleic acid molecules of the invention contain a polynucleotide that encodes a heterologous signal polypeptide to facilitate secretion of TGFβ polypeptides into the culture medium. The choice of signal peptide or can depend on factors such as the type of host cells in which the recombinant polypeptide is to be produced. To illustrate, examples of heterologous signal peptides that are functional in mammalian host cells include the following: the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP 367,566; the type I interleukin-1 receptor signal peptide described in U.S. Pat. No. 4,968,607; and the type II interleukin-1 receptor signal Peptide described in EP 460,846. A leader sequence that has been shown to be effective in insect cells is the lobster tropomyosin leader sequence (Sano et al., 2002, FEBS Lett. 532:143-146). For yeast secretion the signal sequence can be, e.g., the yeast invertase leader, alpha factor leader (including Saccharomyces and Kluyveromyces alpha-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression, mammalian signal sequences can be used to direct secretion of the protein, such as signal sequences from secreted polypeptides of the same or related species, as well as viral secretory leaders.

Preferred embodiments of the invention contain a polynucleotide that encodes a serum albumin signal polypeptide, and more preferably the rat serum albumin signal polypeptide having the following sequence (SEQ ID NO:5): MKWVTFLLLL FISGSFS

Purification of Pro-TGFβ Polypeptide Family Members

A number of procedures can be employed when recombinant pro-TGFβ polypeptide is being purified. For example, proteins having established molecular adhesion properties can be reversible fused to the TGFβ protein. With the appropriate ligand, the pro-TGFβ protein can be selectively adsorbed to a purification column and then freed from the column in a relatively pure form. The fused protein is then removed by enzymatic activity. Finally, pro-TGFβ protein could be purified using immunoaffinity columns.

The TGFβ proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of TGFβ protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Human TGFβ proteins are separated from other bacterial proteins by standard separation techniques, e.g., with Ni-NTA agarose resin.

Alternatively, it is possible to purify TGFβ protein from bacteria periplasm. After lysis of the bacteria, when the TGFβ protein exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

In a preferred embodiment of the invention, polynucleotides of the invention encode a pro-TGFβ polypeptide that is fused to a purification tag polypeptide. As used herein, “purification tag polypeptide” refers any polypeptide sequence that facilitates purification of the mature TGFβ polypeptide with an affinity purification system. The nature of the purification tag polypeptide will depend on the particular affinity purification system used. Various systems are available. In one embodiment, the affinity chromatographic system is immobilized metal affinity chromatography (INIAC), which is based on binding of a purification tag polypeptide to a metal ion resin. Metal ions can be, e.g., zinc, nickel, or cobalt ions. The tag can be a polyhistidine sequence, which interacts specifically with metal ions such as nickel, cobalt, iron, or zinc. A polyhistidine tag can be 2×His; 3×His; 4×His; 5×His; 6×His; 7×His; 8×His or other, provided that it binds essentially specifically to a metal ion. The tag can also be a polylysine or polyarginine sequence, comprising at least four lysine or four arginine residues, respectively, which interact specifically with zinc, copper or a zinc finger protein.

Generally, using IMAC, affinity tagged recombinant proteins are expressed and purified (>90% pure or more) using a one-step purification procedure from crude lysates or cell culture supernatants. Briefly, cells are lysed under native or denaturing conditions (membrane proteins, inclusion bodies, and the like) applied to 96 well plates, and incubated with metal-chelate affinity resin. The resin is washed to remove nonspecifically bound contaminants, and the protein of interest is eluted using either increased imidazole concentrations or low pH.

Commercially available systems for IMAC are known to those of skill in the art include systems that are available from Qiagen, Clontech, InVitrogen and Novagen. Polyhistidine tagged proteins can be purified on nickel affinity chromatography as described in Example 2 below and as described as follows. Ni-agarose beads are equilibrated by washing twice with a 5 times volume of binding buffer, e.g., 50 mM Hepes pH 7.5, 500 mM NaCl, 5% glycerol. The binding buffer can also be 50 mM Tris, pH 7.5, 150 mM NaCl, 2.5 mM MgCl₂. The binding buffer can also be combinations of the two buffers described, or have yet different ingredients, which a person of skill in the art can readily determine. The supernatant of the harvested medium is added to the equilibrated Ni-agarose beads. The non-specifically bound proteins can be removed with a wash buffer, which can be the same as the binding buffer. Additionally, the salt concentration can be increased, e.g., from 150 mM NaCl in the binding buffer to 300 mM NaCl in the wash buffer. Bound protein is eluted with elution buffer, which can be identical to the binding buffer with the addition of about 0 M to about 1 M imidazole, preferably about 0 mM to about 300 mM imidazole and most preferably about 200 mM imidazole. If desired, protein concentrations can be estimated by using the Bio-Rad® protein assay and protein purity can be assessed by SDS-PAGE and Coomassie blue staining. The protein samples can be flash-frozen and stored at −80° C.

Other purification tag polypeptides can comprise an epitope to which an anti-tag antibody can selectively bind. The presence of such epitope-tagged forms of the pro-TGFβ polypeptide can be detected using an antibody against the tag polypeptide. Also, provision of the epitope tag enables the pro-TGFβ polypeptide to be readily purified by affinity purification using an anti-tag antibody or another type of affinity matrix that binds to the epitope tag. Various epitope tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., 1988, Mol. Cell. Biol. 8:2159-2165); the c-myc tag and the 8F9, 3C7, 6E10, G4, B7 and 9E10 antibodies thereto (Evan et al., 1985, Molecular and Cellular Biology, 5:3610-3616); and the Herpes Simplex virus glycoprotein D (gD) tag and its antibody (Paborsky et al., 1990, Protein Engineering 3(6):547-553). Other tag polypeptides include the Flag-peptide (Hopp et al., 1988, Bio Technology 6:1204-1210); the KT3 epitope peptide (Martin et al., 1992 Science, 255:192-194); an alpha-tubulin epitope peptide (Skinner et al., 1991, J. Biol. Chem. 266:15163-15166); and the T7 gene 10 protein peptide tag (Lutz-Freyermuth et al., 1990, Proc. Natl. Acad. Sci. USA 87:6393-6397).

The TGFβ encoding nucleic acid molecules of the invention can be used to produce high quantities of mature TGFβ in culture. Mature TGFβ polypeptides are secreted from cells as biologically inactive homodimers. The pro-TGFβ is cleaved into the C-terminal mature portion and the N-terminal LAP portion, and the mature TGF remains non-covalently bound with the N-terminal LAP portion after secretion. Mature TGFβ is activated by dissociation from the LAP polypeptide, which can be accomplished in vitro by heat, acid or alkaline treatment, deglycosylation or proteolysis by plasmin (Lawrence et al., 1985, Biochem. Biophys. Res. Commun. 133:1026-1034; Lyons, 1988, J. Cell Biol. 106:1659-1665).

When transfected into appropriate host cells and cultured under appropriate conditions, the polynucleotides of the invention can be employed to produce at least about 15, 18, 20, 22, 24, 26, 28, or 30 mgs of mature TGFβ polypeptide per liter of spent culture medium. As described above, when secreted into the culture medium, mature TGFβ is typically non covalently associated with the LAP polypeptide. As used herein, the term “TGFβ complex” refers to this non-covalently bound complex of mature TGFβ and LAP polypeptide. Subsequent processing steps can be used to purify the mature TGFβ from the culture medium and/or separate the mature TGFβ from the LAP polypeptide. It is contemplated as part of the invention that these steps can be accomplished in either order. That is, the TGFβ complex can be purified from the culture medium and then resolved into its separate components, or the TGFβ complex can be dissociated in the culture medium and with the subsequent purification of the mature TGFβ.

Standard protein separation techniques for purifying TGFβ proteins are also contemplated. Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the TGFβ proteins can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The TGFβ proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

A preferred method to purify the mature TGFβ complex from the culture medium employs the use of a binding agent specific for the purification tag polypeptide as described. The purification tag polypeptide is preferably located on the N-terminal portion of the LAP polypeptide. The non-covalent association of the mature TGFβ polypeptide and the LAP polypeptide allows for the purification of both the mature TGFβ polypeptide and the LAP polypeptide using a binding agent for the isolation tag sequence. Once the mature TGFβ complex is purified from the culture medium, mature TGFβ can be dissociated from LAP using any of the activation methods described above and known in the art (e.g., heat, acid or alkaline treatment, deglycosylation or proteolysis by plasmin). “Activating the TGFβ complex” as used herein refers to any method that results in the dissociation of mature TGFβ from the LAP polypeptide. The separation of mature TGFβ from LAP can be accomplished using any the techniques that are well known in the art to accomplish such separations. A preferred method for the separation is the use of size exclusion chromatography.

The process of the invention including the expression of the polynucleotides of the invention in recombinant eukaryotic cells and subsequent purification steps will typically produce mature TGFβ in an amount of at least 15, 18, 20, 22, 24, 26, 28 or 30 mg of mature TGFβ per liter of spent culture medium in a composition of matter that is at least about 93, 95, 97, or 98 percent pure.

D. Pro-TGFβ Amino Acids, Natural Metabolites, Derivatives, Designed Analogs, and Other Organic Molecules

The TGFβ also include organic molecules having TGFβ activity, as defined and described herein. While polypeptides and proteins are often described as exemplary, it should be understood that TGFβ molecules of the present invention are not limited to those having either conventional amino acid side chains or a polyamide backbone structure.

As noted previously, the present invention contemplates a variety of TGFβ molecules, including proteins, polypeptides, and molecules including amino acid residues, as well as a variety of TGFβ compositions. While one tends to think of the “common” natural amino acids (i.e., those listed in Table 1; see Section A above) as being preferred for use in biological compositions, it is also true that a wide variety of other molecules, including uncommon but naturally occurring amino acids, metabolites and catabolites of natural amino acids, substituted amino acids, and amino acid analogs, as well as amino acids in the “D” configuration, are useful in molecules and compositions of the present invention. In addition, “designed” amino acid derivatives, analogs and mimics are also useful in various compounds, compositions and methods of the present invention, as well as polymers including backbone structures composed of non-amide linkages.

For example, in addition to the L-amino acids listed in Section A above, amino acid metabolites such as homoarginine, citrulline, ornithine, and α-aminobutanoic acid are also useful in molecules and compositions of the present invention.

Further, substituted amino acids which are not generally derived from proteins, but which are known in nature, are useful as disclosed herein, include the following examples: L-canavanine; 1-methyl-L-histidine; 3-methyl-L-histidine; 2-methyl L-histidine; α,ε-diaminopimelic acid (L form, meso form, or both); sarcosine; L-ornithine betaine; betaine of histidine (herzynine); L-citrulline; L-phosphoarginine; D-octopine; o-carbamyl-D-serine; γ-aminobutanoic acid; and β-lysine. D-amino acids and D-amino acid analogs, including the following, are also useful in proteins, peptides and compositions of the present invention: D-alanine, D-serine, D-valine, D-leucine, D-isoleucine, D-alloisoleucine, D-phenylalanine, D-glutamic acid, D-proline, and D-allohydroxyproline, and the like.

The present invention also discloses that an extensive variety of amino acids, including metabolites and catabolites thereof, can be incorporated into molecules which display a TGFβ activity. For example, molecules such as ornithine, homoarginine, citrulline, and α-aminobutanoic acid are useful components of molecules displaying TGFβ activity as described herein.

It should also be appreciated that the present invention encompasses a wide variety of modified amino acids, including analogs, metabolites, catabolites, and derivatives, irrespective of the time or location at which modification occurs. In essence, one can place modified amino acids into three categories: (1) catabolites and metabolites of amino acids; (2) modified amino acids generated via posttranslational modification (e.g., modification of side chains); and (3) modifications made to amino acids via non-metabolic or non-catabolic processes (e.g., the synthesis of modified amino acids or derivatives in the laboratory).

The present invention also contemplates that one can readily design side chains of the amino acids of residue units that include longer or shortened side chains by adding or subtracting methylene groups in either linear, branched chain, or hydrocarbon or heterocyclic ring arrangements. The linear and branched chain structures can also contain non-carbon atoms such as S, O, or N. Fatty acids can also be useful constituents of TGFβ molecules herein. The designed side chains can terminate with (R′) or without (R) charged or polar group appendages.

In addition, analogs, including molecules resulting from the use of different linkers, are also useful as disclosed herein. Molecules with side chains linked together via linkages other than the amide linkage, e.g., molecules containing amino acid side chains or other side chains (R— or R′—) wherein the components are linked via carboxy- or phospho-esters, ethylene, methylene, ketone or ether linkages, to name a few examples, are also useful as disclosed herein. In essence, any amino acid side chain, R or R′ group-containing molecule can be useful as disclosed herein, as long as the molecule includes alternating hydrophilic and hydrophobic residues (i.e., component molecules) and displays TGFβ activity as described herein.

The present invention also contemplates molecules comprising peptide dimers joined by an appropriate linker, e.g., peptide dimers linked by cysteine molecules. (As those of skill in the art are aware, two cysteine molecules can be linked together by a disulfide bridge formed by oxidation of their thiol groups.). Such linkers or bridges can thus cross-link different polypeptide chains, dimers, trimers, and the like. Other useful linkers which can be used to connect peptide dimers and/or other peptide multimers include those listed above, e.g., carboxy- or phospho-ester, ethylene, methylene, ketone or ether linkages, and the like.

While it is appreciated that many useful polypeptides disclosed herein, e.g., the TGFβ polypeptides (e.g., SEQ ID NO:1, SEQ ID NO:2, SEQ ID NO:3, and SEQ ID NO:4), comprise naturally-occurring amino acids in the “L” form which are joined via peptide linkages, it should also be understood that molecules including amino acid side chain analogs, non-amide linkages (e.g., differing backbones) can also display a significant TGFβ activity and can possess other advantages, as well. For example, if it is desirable to construct a molecule (e.g., for use in a TGFβ composition) that is not readily degraded, one can wish to synthesize a polypeptide molecule comprising a series of D-amino acids.

“Polypeptoids” are a class of non-natural, sequence-specific polymers representing an alternative derivative of a peptide backbone. Structurally, they differ from polypeptides in that their sidechains are pendant groups of the amide nitrogen rather than the α-carbon (see, e.g., Simon et al., 1992, Proc. Natl. Acad. Sci. U.S.A. 89:9367-9371 and Zuckermann et al., 1992, J. Am. Chem. Soc. 114:10646-10647). “Retropeptoids” are believed to have a higher probability of bioactivity when protein binding is required, as the relative positioning of sidechains and carbonyls “line up” more closely with peptides (Kruijtzer, 1995, Tetrahedron Letters 36:6969-72). N-Substitution prevents proteolysis of the peptoid backbone (see, e.g., Miller et al., 1995, Drug Dev Res 35:20-32), giving enhanced biostability. Since polypeptoids are not proteolyzed, they are not strongly immunogenic (Borman, 1998, C & E News 76:56-57).

In another variation, one can wish to construct a molecule that adopts a more “rigid” conformation; one means of accomplishing this would be to add methyl or other groups to the a carbon atom of the amino acids.

As noted above, other groups besides a CH₃ group can be added to the a carbon atom, that is, TGFβ molecules of the present invention are not limited to those incorporating a CH₃ at the a carbon alone. For example, any of the side chains and molecules described above can be substituted for the indicated CH₃ group at the a carbon component.

As used herein, the terms “analogs” and “derivatives” of polypeptides and amino acid residues are intended to encompass metabolites and catabolites of amino acids, as well as molecules which include linkages, backbones, side-chains or side-groups which differ from those ordinarily found in what are termed “naturally-occurring” L-form amino acids. (The terms “analog” and “derivative” can also conveniently be used interchangeably herein.). Thus, D-amino acids, molecules which mimic amino acids and amino acids with “designed” side chains (i.e., that can substitute for one or more amino acids in a molecule having TGFβ activity) are also encompassed by the terms “analogs” and “derivatives” herein.

A wide assortment of useful TGFβ molecules, including amino acids having one or more extended or substituted R or R′ groups, is also contemplated by the present invention.

Again, one of skill in the art should appreciate that one can make a variety of modifications to individual amino acids, to the linkages, and/or to the chain itself, which modifications will produce molecules falling within the scope of the present invention, as long as the resulting molecule possesses TGFβ activity as described herein.

Having now generally described the invention, the same will be more readily understood through reference to the following examples, which are provided by way of illustration and are not intended to be limiting of the present invention unless specified.

### Exemplary Embodiments

### Example 1

### The Design of the CHO TGFβ1 Expression Construct

Materials and Methods

Chemicals, reagents, and enzymes. Xma I and Bsp 119I can be obtained from Fermetas (Hanover, Md.). BamH I, Xho I and T4 ligase can be obtained from New England Biolabs (Beverly, Mass.). Pfu DNA polymerase can be obtained from Stratagene (Cedar Creek, Tex.).

Construction of the plasmids. An expression plasmid is constructed that contains a porcine TGFβ1 cDNA with several modifications. Porcine TGFβ1 cDNA encodes a precursor TGFβ1 protein that is 94% identical to the human precursor TGFβ1 protein and 100% identical to human mature TGFβ1 The modifications include the introduction of a Cys to Ser amino acid substitution at amino acid residue 33, the replacement of the leader sequence in the porcine cDNA with the leader sequence from rat serum albumin (MKWVTFLLLL FISGSAFS) (SEQ ID NO:5), and the insertion of an 8-histidine tag immediately after the leader sequence. Since the tag is fused to the latency peptide, it does not affect the recombinant mature TGFβ1 amino acid sequence, but enables the use a Ni-NTA based metal affinity chromatography for easy purification of the recombinant pro-TGFβ1 polypeptide. An expression plasmid can be constructed using pcDNA3.1 (+) in which the neomycin gene is replaced with that of glutamine synthetase to enable the amplification of recombinant DNA.

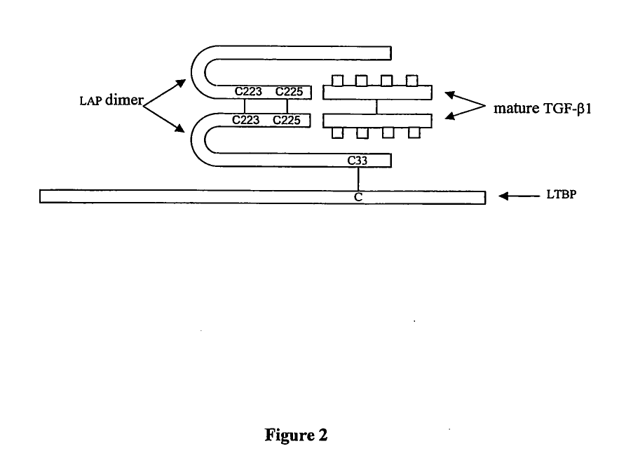

To construct a vector containing glutamine a synthetase (GS) cDNA sequence, a nucleic acid encoding Chinese hamster glutamine synthetase can be amplified by PCR using a CHO-K1 cDNA library (Stratagene, Tex.) as a template. Forward and reverse primers that can be used are as follows: 5′-GCG ATA CCC GGG TAT ACC ATG GCC ACC TCA GCA AGT-3′ (SEQ ID NO:6) and 5′-CGG GTG TTC GAA TTA GTT TTT GTA TTG GAA GGG-3′ (SEQ ID NO:7). Underlined nucleotides of the primers denote the Xma I and Bsp 1191 sites respectively. The PCR product is digested with Xma I and Bsp 119I restriction enzymes and then ligated to the pcDNA3.1 (+) plasmid (Invitrogen, CA), which has been previously digested with these two enzymes. Thus the neomycin gene in pcDNA3.1 (+) is replaced by GS ORF between Xma I and Bsp 119I, permitting amplification of the vector using MSX. This vector, designated pcDNA-GS, is shown in FIG. 2.

The TGFβ1 cDNA, modified to contain the leader sequence from rat serum albumin (RSA), is then inserted into the pcDNA-GS vector to create a plasmid designated pcDNA-GS-TGFβ1. The modified TGFβ1 cDNA can be constructed as follows. A porcine

TGFβ1 cDNA, in which Cys 223 and Cys 225 were mutated to serines, can be obtained from Dr. Seong-Jin Kim, National Institutes of Health, Maryland, and used as a template to amplify a TGFβ1 fragment in two PCR reactions using the following primers: TGF1-(5′-GGT TCT GCC TTT TCT CAC CAC CAT CAC CAC CAC CAT CAT CTG TCC ACC TGC AAG AC-3′) (SEQ ID NO:8); TGF2-(5′-TAG T CTC GAG TTA TCA GCT GCA CTT GCA GG-3′) (SEQ ID NO:9); RSA1: -(5′-AAA GGG GGA TCC GCC ACC ATG AAG TGG GTA ACC TTT CTC CTC CTC-3′) (SEQ ID NO:10); RSA2-(5′-AGA AAA GGC AGA ACC GGA GAT GAA GAG GAG GAG GAG AAA GGT TAC-3′) (SEQ ID NO:11). A first PCR reaction is conducted containing the TGF1, TGF2 primers and the porcine TGFβ1 cDNA. The product of the first PCR reaction is then used as a template for a second PCR reaction using the RSA1, RSA2 and TGF2 primers. The DNA segment amplified from the second PCR reaction encodes, from 5′ end to 3′ end, a rat serum albumin leader sequence, eight histidine residues and porcine pro-TGFβ1. The PCR product also contains a BamH I site at 5′ end and an Xho I site at 3′ end. The PCR product is then digested with BamH I and Xho I and then inserted between BamH I and Xho I sites of pcDNA-GS vector.

The serine residues at position 223 and 225 of TGFβ1 are mutated back to cysteines with two oligonucleotides, 5′-CGC CTC AGT GCC CAC TGT TCC TGT GAC AGC AAA GAT AAC-3′ (SEQ ID NO:12) and 5′-GTT ATC TTT GCT GTC ACA GGA ACA GTG GGC ACT GAG GCG-3′ (SEQ ID NO:13), and the QuikChange Site-Directed Mutagenesis kit (Stratagene, Tex.) A further mutation is introduced to replace Cys 33 of TGFβ1 with serine using primers: 5′-GGA TCC CTG TCC ACC TCC AAG ACC ATC GAC ATG-3′ (SEQ ID NO:14) and 5′-CAT GTC GAT GGT CTT GGA GGT GGA CAG GGA TCC-3′ (SEQ ID NO:15). The resulting expression vector is confirmed by DNA sequencing and designated pcDNA-GS-TGFβ1.

### Example 2

### Expression and Purification of TGFβ1

Material and Methods

TGFβ1 content and protein concentration assays. TGFβ1 content is measured by ELISA using a DuoSet ELISA Development kit (R & D Systems, Minneapolis, Minn.). A sample of 0.2 μg of mouse anti-human TGFβ1 antibody is immobilized onto each well of ELISA plate. A chicken anti-human TGFβ1 antibody is used as the detection antibody at a concentration of 300 ng/ml. Samples are acid activated prior to the assay, and the assay is performed according to the instruction manual. Protein concentration is determined by using a Bicinchoninic Acid (BCA) protein assay kit (Sigma-Aldrich, MO).

SDS-PAGE and Western blot assays. SDS-PAGE is performed using a Homogeneous 20% PhastGel on PhastSystem (Amersham Biosciences, NJ) or a 12.5% SDS-polyacrylamide ready gel (Bio-Rad Laboratories, Hercules, Calif.). The gels are stained with Coomassie brilliant blue or silver, according to manufacturer's specifications. Western blot assays are performed as described by Sambrook et al., 1989, in Detection and analysis of proteins expressed from cloned genes, in Molecular Cloning: A laboratory Manual, 2nd edition, 18.64-18.70. A mouse anti-human TGFβ1 antibody (R&D Systems, Minneapolis, Minn.) at a concentration of 2 μg/ml is the primary antibody and a goat anti-mouse IgG-horseradish peroxidase conjugate (Bio-Rad Laboratories, CA). at a 1:20000 dilution is the secondary antibody. The peroxidase substrate is 3-Amino-9-ethylcarbazole (Sigma-Aldrich, MO).

Results

Expression of the modified TGFβ1 cDNA of example 1 above is achieved through introduction of the pcDNA-GS-TGFβ plasmid into Chinese hamster ovary cells (hereinafter “CHO-cells”). Cells of the CHO cell line, CHO-lec 3.2.8.1, (available from Dr. Pamela Stanley, Albert Einstein College of Medicine, NY) are trypsinized and seeded in a T-25 flask. Prior to transfection, cells are grown under non-selective conditions in DMEM/F12 (Invitrogen, Carlsbad, Calif.), containing 5% fetal bovine serum (FBS) for approximately twenty-four hours, until they were almost confluent. Immediately prior to transfection, the medium is replaced with 4 ml fresh DMEM/F12 containing 5% FBS medium. A 10 μg sample of the plasmid, pcDNA-GS-TGFβ1 and 30 μl aliquot of the Lipofectamine 2000 reagent (Invitrogen, CA) are each diluted into 0.5 ml OPTI-MEM I medium separately, and then the dilutions are combined to incubate at room temperature for 20 minutes. The mixture is then added to the CHO cells and the cells are incubated. After 24 hours, the medium is replaced with fresh medium. Two days post-transfection, the cells are trypsinized and seeded into ten 10 cm culture dishes, and cultured in selection media (Glutamine-Free GMEM-S (JRH Biosciences, Lenexa, Kans.) supplemented with GS supplement, 5% Dialyzed FBS and 30 μM Methionine sulfoximine (MSX) (Sigma-Aldrich, St Louis, Mo.)). After about 3 weeks, colonies are transferred into 48-well plates containing 0.5 ml selection medium. When the cultures reach confluency, supernatants are taken and assayed for TGFβ1 by ELISA. The ten clones showing the highest expression levels are subjected to another round of selection by further splitting of the clones into 10 cm dishes containing 150 μM MSX selection medium. A third round of selection is then made in the presence of 500 μM MSX. After three rounds of selection and amplification, the clone with highest TGFβ1 expression is chosen for large-scale recombinant protein production.

To generate TGFβ1 containing medium, the chosen clone is transferred to a T-500 triple layer flasks after expansion and cultured in Glutamine-Free GMEM-S, supplemented with GS supplement, 5% Dialyzed FBS and 500 μM MSX. When the cultures reach confluency, cells are washed twice with Hank's solution and cultured with CHO—S—SFM II serum free medium (Invitrogen, Carlsbad, Calif.). After three to four days, the medium is harvested and replaced with fresh serum free medium. The concentration of mature TGFβ1 in the harvested medium, as determined by ELISA assay, is about 30 mg/liter.

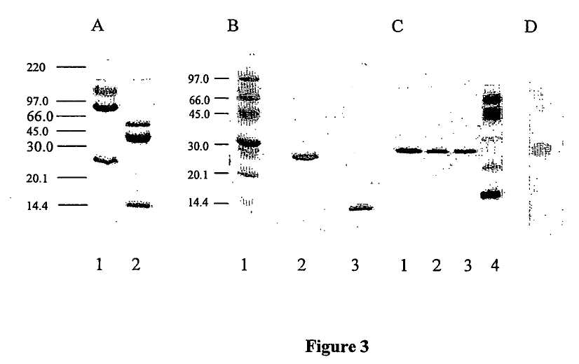

Due to the high and specific affinity of His-tag with Ni-NTA, the harvest medium can be directly loaded onto the Ni-NTA column without a prior concentration step. The harvested medium is filtered with a 0.22 μm cellulose acetate filter and stored at −20° C. for future use. A 20 ml Ni-NTA agarose (Qiagen, Valencia, Calif.) column is equilibrated with a loading buffer (50 mM Tris, 150 mM sodium chloride, pH 8.0). Approximately 500 ml harvested medium is thawed and filtered again with 0.22 μm cellulose filter and then directly applied to the Ni-NTA column. After loading, the column is washed with 30 ml of the loading buffer and the protein is eluted by a linear concentration gradient of imidazole from 0.0 M to 0.3 M, with 45 ml between the loading and elution buffer (50 mM Tris, 150 mM sodium chloride and 1M imidazole, pH 8.0). After the first step of purification, the eluted protein mainly consists of pro-TGFβ1, giving rise to three major bands on SDS-PAGE that represented pro-TGFβ1 (the uppermost band), LAP (the middle band), and mature TGFβ1 (the lower band) (FIG. 3, Panel A) Sequencing of the N-terminus of pro-TGFβ1 and LAP results in a peptide of HHHHHHHHLSTSKTIDMELV (SEQ ID NO:16), indicating that the leader sequence is cleaved off right before the histidine tag.

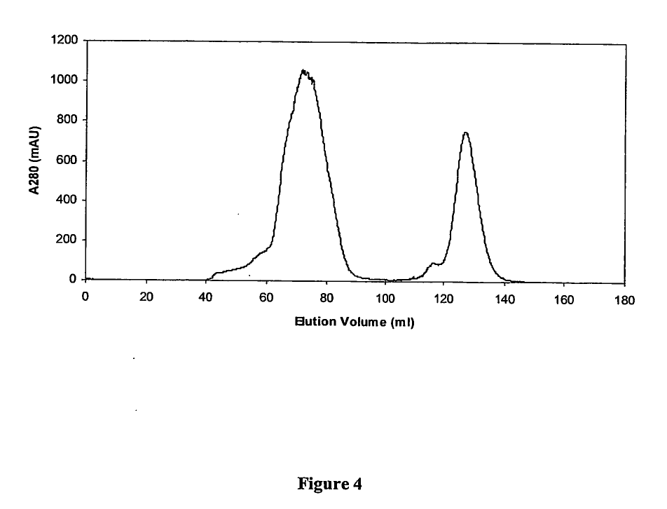

Ni-NTA affinity eluted TGFβ1 is pH adjusted to 3.0 to release the mature TGFβ1 (25 KD) from its latency protein LAP (60-75 KD). The mature TGFβ1 is then purified to homogeneity using a size exclusion column. The sample is concentrated two fold, filtered, and applied several times to a HiLoad 16/60 Superdex 200 prep grade column (Amersham Biosiences, Piscataway, N.J.) with a running buffer (50 mM Glycine, 50 mM sodium chloride, pH 4.0). Samples from gel filtration peak fractions are taken for analysis (FIG. 3, Panels B and C, FIG. 4). The elution profile shows that TGFβ1 eluted at a smaller apparent molecular weight (FIG. 4). This phenomenon has also been observed by other groups and could be due to the hydrophobic interaction between TGFβ1 and the chromatographic matrix. (Gentry et al., 1988, Molecular events in the processing of recombinant type 1 pre-pro-transforming growth factor beta to the mature polypeptide, Mol. Cell. Biol. 8: 4162-4168).

After only two steps of purification, 10 mg purified TGFβ1 is obtained from 500 ml harvest medium. The identity of the purified mature TGFβ1 polypeptide is further confirmed by Western blot assay (FIG. 3, Panel D), N-terminal sequencing (ALDTNYCFSSTEKNCCVRQL) (SEQ ID NO:17), and mass spectrometry (Mr. 25574.0).

### Example 3

### Binding Between TGFβ1 and its Receptors

The binding of TGFβ1 with the TGFβ1 Type II receptor (TβRII) is assessed in comparison the binding of commercial TGFβ1. Two methods can be used this comparison: (1) an immunoassay technique and (2) surface plasmon-resonance measurements.

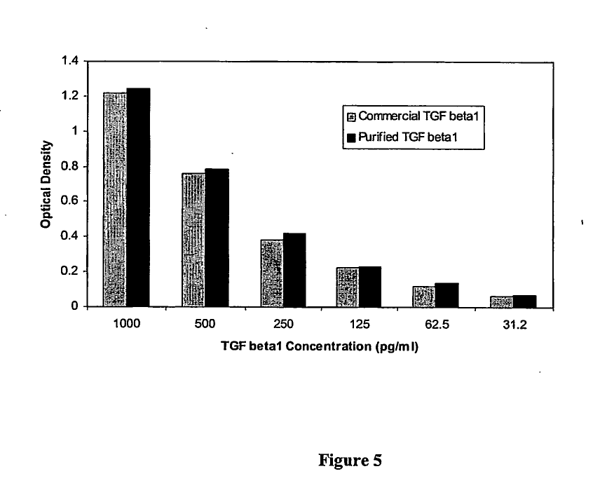

For the immunoassay technique, Quantikine Human TGFβ1 Immunoassay kit (R & D Systems, Minneapolis, Minn.) is employed. This kit has recombinant human TβRII coated on a substrate and provides a TGFβ1 standard. This test is performed according to the instruction protocol and the binding of TGFβ1 to the substrate is assessed in comparison with the TGFβ1 standard. As shown in FIG. 5, the results from this comparison show that the binding affinity of the purified TGFβ1 to TβRII is equal to that of the TGFβ1 standard.

Surface plasmon resonance (SPR) measurements are performed using a BIAcore 3000 instrument (BIAcore, Uppsala, Sweden). TGFβ1 is immobilized on two flow cells of a CM5 sensor chip at concentrations of 0.7 μM and 1.4 μM respectively, in 50 mM sodium acetate, pH 5.0, using N-hydrosuccinimide/1-ethyl-3 (−3-dimethylaminopropyl)-carbodiimide hydrochloride (NHS/EDC) at a flow rate of 20 μl/min.

TGFβ1 type II receptor (residues 22-136) is prepared essentially as described in Boesen, 2000, Protein Expr. Purif. 20:98-104. In brief, a 25 mg sample of the inclusion bodies is dissolved in 6M guanidine hydrochloride and then injected into a 1 L refolding solution (0.5 M arginine, 20 mg/L AEBSF protease inhibitor, 2 mM EDTA, 5 mM cysteamine (reduced form), 0.5 mM cystamine (oxidized form), and 0.1 M Tris, pH8.0). The solution is stirred for 24 hours at 10° C. and then dialyzed against dialysis buffer (5 mM Tris, pH8.5). After filtering, the refolding solution is loaded onto Source 15Q column (Amersham Biosiences, NJ), which is pre-equilibrated with a running buffer (5 mM Tris, pH8.5). Two major protein peaks are eluted by a linear gradient from 0 to 1 M sodium chloride. The first peak fractions are pooled and concentrated and then purified with a Superdex 200 column.

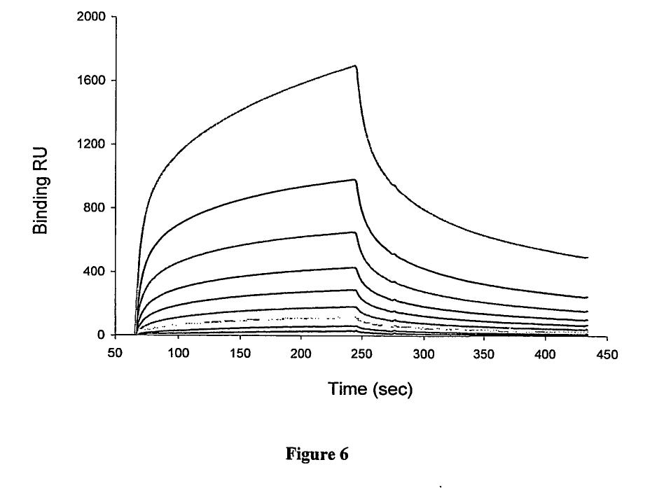

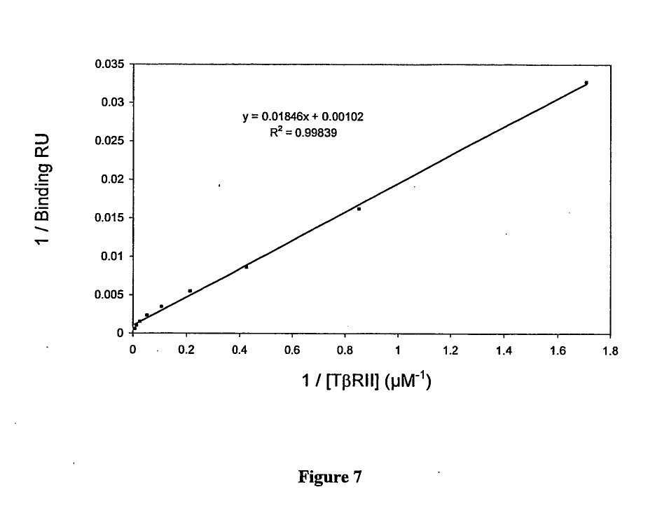

Binding of the type II TGFβ receptor (TβRII) to the immobilized TGFβ1 is measured using serial dilution of TβRII from 150 nM to 0.6 nM at a flow rate of 20 μl/min. The running buffer and the analyte sample buffer are 10 mM Tris hydrochloride, pH 8.0. The chip surface is regenerated with 10 mM glycine, pH 3.0, at a flow rate of 30 μl/min for 30 seconds, followed by buffer stabilization for 3 minutes. The apparent dissociation constant (K_{D}) is obtained from a linear regression of steady state 1/Response versus 1/C plots. Surface plasmon resonance measurements indicate an apparent dissociation constant (K_{D}) of TGFβ1 with human type II receptor was about 55 nM (FIG. 6 and FIG. 7).

### Example 4

TGFβ1 Biological Activity

In order to determine whether the purified TGFβ1 was biologically active, and to compare its activity with commercial TGFβ1, a bioassay is conducted which assesses the ability of TGFβ1 to induce luciferase expression in mink-lung-epithelial cells (MLEC) stably transfected with an expression construct comprising a truncated plasminogen activator inhibitor-1 promoter fused to the firefly luciferase reporter gene (Abe et al., 1994, Anal. Biochem. 216:276-284).

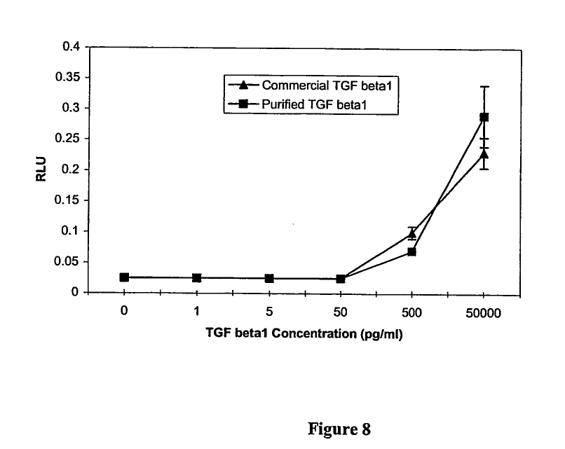

Transfected MLECs are plated in 96-well tissue culture dishes at a concentration of 6×10⁴ cells per well. Cells are allowed to attach for 3 hours at 37° C. in a 5% CO₂ incubator and then the medium is replaced with test samples containing varying concentrations of TGFβ1 diluted in DMEM+0.1% BSA. After 14 hours at 37° C., cell extracts are prepared and assayed for luciferase activity using the enhanced luciferase assay kit (Analytical Luminescence, San Diego, Calif.) as per the manufacturer's instructions. Luciferase activity is reported as relative light units (RLU). Commercial TGFβ1 (R & D Systems, MN) is used as a comparison. The dose-response curves show that the biological activities of the purified TGFβ1 and commercial TGFβ1 are comparable (FIG. 8).

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

All publications and patent documents cited above are hereby incorporated by reference in their entirety for all purposes to the same extent as if each were so individually denoted.

Each recited range includes all combinations and sub-combinations of ranges, as well as specific numerals contained therein.

What is claimed:

1. An isolated mammalian TGFβ-encoding nucleic acid comprising:
(a) a pro-TGFβ polynucleotide encoding a mammalian pro-TGFβ polypeptide, wherein the polynucleotide does not encode a cysteine residue within the first ten amino acid residues of the pro-TGFβ polypeptide; and
(b) a signal polynucleotide encoding a heterologous signal polypeptide, wherein the signal polynucleotide is in frame with the pro-TGFβ polynucleotide.

2. An isolated mammalian TGFβ-encoding nucleic acid according to claim 1 wherein the pro-TGFβ polynucleotide encodes a mammalian pro-TGFβ polypeptide comprising a mature TGFβ portion and a LAP portion, wherein the mature TGFβ portion is 95% identical to a mature human TGFβ molecule.

3. An isolated mammalian TGFβ-encoding nucleic acid according to claim 2 wherein the pro-TGFβ polynucleotide is selected from the group consisting of:
(a) a pro-TGFβ polynucleotide encoding a pro-TGFβ polypeptide, wherein the mature TGFβ portion is identical to mature human TGFβ1;
(b) a pro-TGFβ polynucleotide encoding a pro-TGFβ polypeptide, wherein the mature TGFβ portion is identical to mature human TGFβ2;
(c) a pro-TGFβ polynucleotide encoding a pro-TGFβ polypeptide, wherein the mature TGFβ portion is identical to mature human TGFβ3;
(d) a pro-TGFβ polynucleotide encoding a pro-TGFβ polypeptide, wherein the mature TGFβ portion is identical to mature human TGFβ1, and wherein the LAP portion is at least 90% identical to the LAP portion of human pro TGFβ1;
(e) a pro-TGFβ polynucleotide encoding a pro-TGFβ polypeptide, wherein the mature TGFβ portion is identical to mature human TGFβ2, and wherein the LAP portion is at least 90% identical to the LAP portion of human pro TGFβ2; and
(f) a pro-TGFβ polynucleotide encoding a pro-TGFβ polypeptide, wherein the mature TGFβ portion is identical to mature human TGFβ3, and wherein the LAP portion is at least 90% identical to the LAP portion of human pro TGFβ3.

4. An isolated nucleic acid molecule according to claim 3, further comprising a tag polynucleotide encoding a purification tag polypeptide, wherein the tag polynucleotide is located between, and in frame with, the signal polynucleotide and the pro-TGFβ polynucleotide.

5. An isolated eukaryotic cell line comprising the isolated nucleic acid molecule of claim 4.

6. A vector comprising the isolated mammalian TGFβ-encoding nucleic acid molecule of claim 4.

7. An expression vector comprising the isolated mammalian TGFβ-encoding nucleic acid molecule of claim 4.

8. The expression vector of claim 7, wherein the nucleic acid is operatively linked to the regulatory sequence in an antisense orientation.

9. The expression vector of claim 8, wherein the polynucleotide is operatively linked to the regulatory sequence in a sense orientation.

10. A host cell comprising the nucleic acid of claim 4, or progeny of the cell.

11. The host cell of claim 10, which is a eukaryote.

12. The host cell of claim 11, wherein the nucleic acid is operatively linked to the regulatory sequence in an antisense orientation.

13. The polynucleotide of claim 4 that is RNA.

14. An isolated polypeptide encoded by a nucleic acid of claim 1.

15. The polypeptide of claim 14 that has the amino acid sequence of SEQ ID NO:1, SEQ ID NO:2, SEQ ID NO:3, or SEQ ID NO:4.

16. The isolated polypeptide of claim 14 that is fused with a heterologous peptide.

17. A method of producing mature TGFβ polypeptide comprising culturing an isolated eukaryotic cell line according to claim 5 in culture medium under conditions wherein greater than 25 mg of mature TGFβ per liter of culture medium is produced; and recovering the TGFβ polypeptide from the isolated cell line or its medium.

18. A method of producing mature TGFβ polypeptide comprising:
(a) culturing an isolated eukaryotic cell line according to claim 5 in culture medium under conditions to produce TGFβ complex in the culture medium, wherein TGFβ complex comprises mature TGFβ polypeptide and LAP polypeptide fused with a purification tag polypeptide;
(b) purifying the TGFβ complex by binding the TGFβ complex with a binding agent that specifically binds the purification tag polypeptide;
(c) activating the TGFβ complex to dissociate mature TGFβ from associated LAP polypeptide; and
(d) separating mature TGFβ polypeptide from the LAP polypeptide; and
(e) recovering the TGFβ polypeptide from the isolated cell line or its medium.

19. A method of producing mature TGFβ polypeptide according to claim 18, wherein purified mature TGFβ is produced with a yield of greater than 15 mg per liter of culture medium and a purity of greater than 98%.

20. An isolated Chinese hamster ovary cell line comprising a pro-TGFβ polynucleotide encoding a mammalian pro-TGFβ polypeptide, wherein the polynucleotide does not encode a cysteine residue within the first ten amino acid residues of the pro-TGFβ polypeptide, or progeny of the cell line.

21. A method of producing mature TGFβ polypeptide comprising culturing an isolated eukaryotic cell line according to claim 20 in culture medium under conditions wherein greater than 25 mg of mature TGFβ per liter of culture medium is produced; and recovering the TGFβ polypeptide from the isolated cell line or its medium.

22. A method of producing mature TGFβ polypeptide comprising culturing an isolated eukaryotic cell line comprising a recombinant pro-TGFβ polynucleotide encoding a mammalian pro-TGFβ polypeptide, wherein the polynucleotide does not encode a cysteine residue within the first ten amino acid residues of the pro-TGFβ polypeptide, and wherein the cell line is cultured under conditions that produce greater than 25 mg of mature TGFβ per liter of culture medium; and recovering the TGFβ polypeptide from the isolated cell line or its medium.
